(19) United States
(12) Patent Application Publication
(10) Pub. No.: US 2020/0061380 A1
(43) Pub. Date: Feb. 27, 2020

(54) Stimulation Using Long Duration Waveform Phases in a Spinal Cord Stimulator System

(71) Applicant: Boston Scientific Neuromodulation Corporation, Valencia, CA (US)

(72) Inventors: Tianhe Zhang, Studio City, CA (US); Rosana Esteller, Santa Clarita, CA (US); Michael A. Moffitt, Saugus, CA (US); Rafael Carbunaru, Valley Village, CA (US)

(21) Appl. No.: 16/537,279

(22) Filed: Aug. 9, 2019

Related U.S. Application Data
(60) Provisional application No. 62/721,992, filed on Aug. 23, 2018.

(51) Int. Cl. A61N 1/36

(52) U.S. Cl. CPC A61N 1/36071; A61N 1/37247; A61N 1/36189; A61N 1/36062

(57) ABSTRACT

Disclosed are systems and methods for providing stimulation using waveforms with long duration phases in a spinal cord stimulator. Simulation shows the effectiveness of using phase durations of greater than 2.0 ms, or even 2.6 ms or greater, in recruiting inhibitory interneurons in the dorsal horn of the spinal cord, or in recruiting dorsal column axons of the dorsal column, both of which promote pain suppression in spinal cord stimulation (SCS) patients. Traditional SCS devices may not allow the programming of phase durations of such lengths, and so examples of how long phase durations can be effectively created is shown by way of a non-limiting example, preferably in a single timing channel. The waveforms preferably have at least two phases of opposite polarities, at least one of which is long, although phases may be split into sub-phases. The waveforms may be charge balanced at each electrode.

### FIELD OF THE INVENTION

This is a non-provisional application of U.S. Provisional Patent Application Ser. No. 62/721,992, filed Aug. 23, 2018, to which priority is claimed, and which is incorporated by reference.

### FIELD OF THE INVENTION

This application relates to Implantable Medical Devices (IMDs), and more specifically to techniques for providing stimulation in implantable neurostimulation systems.

### INTRODUCTION

Implantable neurostimulator devices are devices that generate and deliver electrical stimuli to body nerves and tissues for the therapy of various biological disorders, such as pacemakers to treat cardiac arrhythmia, defibrillators to treat cardiac fibrillation, cochlear stimulators to treat deafness, retinal stimulators to treat blindness, muscle stimulators to produce coordinated limb movement, spinal cord stimulators to treat chronic pain, cortical and deep brain stimulators to treat motor and psychological disorders, and other neural stimulators to treat urinary incontinence, sleep apnea, shoulder subluxation, etc. The description that follows will generally focus on the use of the invention within a spinal cord stimulation (SCS) system, such as that disclosed in U.S. Pat. No. 6,516,227. However, the present invention may find applicability with any implantable neurostimulator device system.

An SCS system typically includes an Implantable Pulse Generator (IPG) 10 shown in FIG. 1. The IPG 10 includes a biocompatible conductive device case 12 that holds the IPG's circuitry and a battery 14 for providing power for the IPG to function. The IPG 10 is coupled to tissue-stimulating electrodes 16 via one or more electrode leads that form an electrode array 17. For example, one or more percutaneous leads 15 can be used having ring-shaped or split-ring electrodes 16 carried on a flexible body 18. In another example, a paddle lead 19 provides electrodes 16 positioned on one of its generally flat surfaces. Lead wires 20 within the leads are coupled to proximal contacts 21, which are insertable into lead connectors 22 fixed in a header 23 on the IPG 10, which header can comprise an epoxy for example. Once inserted, the proximal contacts 21 connect to header contacts 24 within the lead connectors 22, which are in turn coupled by feedthrough pins 25 through a case feedthrough 26 to stimulation circuitry 28 within the case 12, which stimulation circuitry 28 is described below.

In the illustrated IPG 10, there are thirty-two electrodes (E1-E32), split between four percutaneous leads 15, or contained on a single paddle lead 19, and thus the header 23 may include a 2×2 array of eight-electrode lead connectors 22. However, the type and number of leads, and the number of electrodes, in an IPG is application specific and therefore can vary. The conductive case 12 can also comprise an electrode (Ec). In a SCS application, the electrode lead(s) are typically implanted in the spinal column proximate to the dura in a patient's spinal cord, preferably spanning left and right of the patient's spinal column. The proximal contacts 21 are then tunneled through the patient's tissue to a distant location such as the buttocks where the IPG case 12 is implanted, where they are coupled to the lead connectors 22. In other IPG examples designed for implantation directly at a site requiring stimulation, the IPG can be lead-less, having electrodes 16 instead appearing on the body of the IPG 10 for contacting the patient's tissue. The IPG lead(s) can be integrated with and permanently connected to the IPG 10 in other solutions. The goal of SCS therapy is to provide electrical stimulation from the electrodes 16 to alleviate a patient's symptoms, such as chronic back pain.

IPG 10 can include an antenna 27a allowing it to communicate bi-directionally with a number of external devices discussed subsequently. Antenna 27a as shown comprises a conductive coil within the case 12, although the coil antenna 27a can also appear in the header 23. When antenna 27a is configured as a coil, communication with external devices preferably occurs using near-field magnetic induction. IPG 10 may also include a Radio-Frequency (RF) antenna 27b. RF antenna 27b is shown within the header 23, but it may also be within the case 12. RF antenna 27b may comprise a patch, slot, or wire, and may operate as a monopole or dipole. RF antenna 27b preferably communicates using far-field electromagnetic waves, and may operate in accordance with any number of known RF communication standards, such as Bluetooth, Zigbee, MICS, and the like.

Stimulation in IPG 10 is typically provided by a sequence of waveforms (e.g., pulses) each of which may include a number of phases such as 30a and 30b, as shown in the example of FIG. 2A. Stimulation parameters typically include amplitude (current A, although a voltage amplitude V can also be used); frequency (f); phase duration (PD) of the phases of the waveform such as 30a and 30b; the electrodes 16 selected to provide the stimulation; and the polarity of such selected electrodes, i.e., whether they act as anodes that source current to the tissue or cathodes that sink current from the tissue. These and possibly other stimulation parameters taken together comprise a stimulation program that the stimulation circuitry 28 in the IPG 10 can execute to provide therapeutic stimulation to a patient.

In the example of FIG. 2A, electrode El has been selected as an anode (during first phase 30a), and thus sources a positive current of amplitude +A to the tissue. Electrode E2 has been selected as a cathode (again during first phases 30a), and thus sinks a corresponding negative current of amplitude −A from the tissue. This is an example of bipolar stimulation, in which only lead-based electrodes are used to provide stimulation to the tissue. However, more than one electrode may be selected to act as an anode at a given time, and more than one electrode may be selected to act as a cathode at a given time. The case electrode may also be selected as an anode or cathode along with one or more lead-based electrodes, in what is known as monopolar stimulation.

IPG 10 as mentioned includes stimulation circuitry 28 to form prescribed stimulation at a patient's tissue. FIG. 3 shows an example of stimulation circuitry 28, which includes one or more current sources 40, and one or more current sinks 42ᵢ. The sources and sinks 40ᵢ and 42ᵢ can comprise Digital-to-Analog converters (DACs), and may be referred to as PDACs 40ᵢ and NDACs 42ᵢ in accordance with the Positive (sourced, anodic) and Negative (sunk, cathodic) currents they respectively issue. In the example shown, a NDAC/PDAC 40ᵢ/42ᵢ pair is dedicated (hardwired) to a particular electrode node ei 39. Each electrode node ei 39 is connected to an electrode Ei 16 via a DC-blocking capacitor Ci 38, for the reasons explained below. PDACs 40ᵢ and NDACs 42ᵢ can also comprise voltage sources.

Proper control of the PDACs 40ᵢ and NDACs 42ᵢ allows any of the electrodes 16 and the case electrode Ec 12 to act as anodes or cathodes to create a current through a patient's tissue, R, hopefully with good therapeutic effect. In the example shown, and consistent with the first phase 30a of FIG. 2A, electrode E1 has been selected as an anode electrode to source current I=+A to the tissue R and electrode E2 has been selected as a cathode electrode to sink current I=−A from the tissue R. Thus PDAC 40₁ and NDAC 42₂ are activated and digitally programmed to produce the desired current, I, with the correct timing (e.g., in accordance with the prescribed frequency f and phase durations PD). Power for the stimulation circuitry 28 is provided by a compliance voltage VH, as described in further detail in U.S. Patent Application Publication 2013/0289665.

Other stimulation circuitries 28 can also be used in the IPG 10. In an example not shown, a switching matrix can intervene between the one or more PDACs 40ᵢ and the electrode nodes ei 39, and between the one or more NDACs 42ᵢ and the electrode nodes. Switching matrices allows one or more of the PDACs or one or more of the NDACs to be connected to one or more electrode nodes at a given time. Various examples of stimulation circuitries can be found in U.S. Pat. Nos. 6,181,969, 8,606,362, 8,620,436, and U.S. Patent Application Publications 2018/0071520 and 2019/0083796.

Much of the stimulation circuitry 28 of FIG. 3, including the PDACs 40ᵢ and NDACs 42ᵢ, the switch matrices (if present), and the electrode nodes ei 39 can be integrated on one or more Application Specific Integrated Circuits (ASICs), as described in U.S. Patent Application Publications 2012/0095529, 2012/0092031, 2012/0095519, 2018/0071516, and 2018/0071513, which are incorporated herein by reference in their entireties. As explained in these references, ASIC(s) may also contain other circuitry useful in the IPG 10, such as telemetry circuitry (for interfacing off chip with telemetry antennas 27a and/or 27b), circuitry for generating the compliance voltage VH, various measurement circuits, etc.

Also shown in FIG. 3 are DC-blocking capacitors Ci 38 placed in series in the electrode current paths between each of the electrode nodes ei 39 and the electrodes Ei 16 (including the case electrode Ec 12). The DC-blocking capacitors 38 act as a safety measure to prevent DC current injection into the patient, as could occur for example if there is a circuit fault in the stimulation circuitry 28. The DC-blocking capacitors 38 are typically provided off-chip (off of the ASIC(s)), and instead may be provided in or on a circuit board in the IPG 10 used to integrate its various components, as explained in U.S. Patent Application Publication 2015/0157861.

Referring again to FIG. 2A, the stimulation waveforms as shown are biphasic, with each waveform comprising a first phase 30a followed thereafter by a second phase 30b of opposite polarity. (Although not shown, an interphase period during which no active current is driven may intervene between the phases 30a and 30b, as discussed later). Both of the phases 30a and 30b are actively driven by the stimulation circuitry 28 by causing relevant PDACs 40, and NDACs 42ᵢ to drive the prescribed currents. Biphasic waveforms are useful to actively recover any charge that might be stored on capacitive elements in the current path, such as on the DC-blocking capacitors 38. Charge recovery is shown with reference to both FIGS. 2A and 2B. During the first phases 30a, charge will build up across the DC-blocking capacitors C1 and C2 associated with the electrodes E1 and E2 selected to produce the current, giving rise to voltages Vc1 and Vc2. Given the definition of these voltages in FIG. 2B, they are of the same polarity as shown in FIG. 2A. During the second phases 30b, when the polarity of the current is reversed at the selected electrodes E1 and E2, the stored charge on capacitors C1 and C2 is recovered, and thus voltages Vc1 and Vc2 return to 0V at the end the second phase 30b.

To recover all charge by the end of the second phase 30b of each waveform (Vc1=Vc2=0V), the first and second phases 30a and 30b are charged balanced at each electrode, with the first phase 30a providing a charge of +Q(+A*PD) and the second phase 30b providing a charge of −Q(−A*PD) at electrode E1, and with the first phase 30a providing a charge of −Q and the second phase 30b providing a charge of +Q at the electrode E2. In the example shown, such charge balancing is achieved by using the same phase duration (PD) and the same amplitude (|A|) for each of the opposite-polarity phases 30a and 30b. However, the phases 30a and 30b may also be charged balance at each electrode if the product of the amplitude and phase durations of the two phases 30a and 30b are equal, or if the area under each of the phases (their integrals) is equal, as is known.

FIG. 3 shows that stimulation circuitry 28 can include passive recovery circuitry, which is described further in U.S. Patent Application Publications 2018/0071527 and 2018/0140831. Specifically, passive recovery switches 41ᵢ may be attached to each of the electrode nodes ei 39, and are used to passively recover any charge remaining on the DC-blocking capacitors Ci 38 after issuance of the second phase 30b—i.e., to recover charge without actively driving a current using the DAC circuitry. Passive charge recovery can be prudent, because non-idealities in the stimulation circuitry 28 may lead to phases 30a and 30b that are not perfectly charge balanced.

Therefore, and as shown in FIG. 2A, passive charge recovery typically occurs after the issuance of second phases 30b, for example during at least a portion 30c of the quiet periods between the waveforms, by closing passive recovery switches 41ᵢ. As shown in FIG. 3, the other end of the switches 41ᵢ not coupled to the electrode nodes ei 39 are connected to a common reference voltage, which in this example comprises the voltage of the battery 14, Vbat, although another reference voltage could be used. As explained in the above-cited references, passive charge recovery tends to equilibrate the charge on the DC-blocking capacitors 38 by placing the capacitors in parallel between the reference voltage (Vbat) and the patient's tissue. Note that passive charge recovery is illustrated as small exponentially-decaying curves during 30c in FIG. 2A due to the R-C nature of the circuit, and this current may be positive or negative depending on whether phase 30a or 30b has a predominance of charge at a given electrode.

FIG. 4 shows an external trial stimulation environment that may precede implantation of an IPG 10 in a patient. During external trial stimulation, stimulation can be tried on a prospective implant patient without going so far as to implant the IPG 10. Instead, one or more trial electrode arrays 17′ (e.g., one or more trial percutaneous leads 15 or trial paddle leads 19) are implanted in the patient's tissue at a target location 52, such as within the spinal column as explained earlier. The proximal ends of the trial electrode array(s) 17′ exit an incision 54 and are connected to an External Trial Stimulator (ETS) 50. The ETS 50 generally mimics operation of the IPG 10, and thus can provide stimulation to the patient's tissue via its stimulation circuitry 58, which may be equivalent or identical to stimulation circuitry 28 in the IPG 10. The ETS 50 is generally worn externally by the patient for a short while (e.g., two weeks), which allows the patient and his clinician to experiment with different stimulation parameters to hopefully find a stimulation program that alleviates the patient's symptoms (e.g., pain). If external trial stimulation proves successful, the trial electrode array(s) 17′ are explanted, and a full IPG 10 and a permanent electrode array 17 (e.g., one or more percutaneous 15 or paddle 19 leads) are implanted as described above; if unsuccessful, the trial electrode array(s) 17′ are simply explanted.

Like the IPG 10, the ETS 50 can include one or more antennas to enable bi-directional communications with external devices such as those shown in FIG. 5. Such antennas can include a near-field magnetic-induction coil antenna 56a, and/or a far-field RF antenna 56b, as described earlier. ETS 50 may also include a battery (not shown) for operational power.

FIG. 5 shows various external devices that can wirelessly communicate data with the IPG 10 and the ETS 50, including a patient hand-held external controller 60, and a clinician programmer 70. Both of devices 60 and 70 can be used to wirelessly transmit a stimulation program to the IPG 10 or ETS 50—that is, to program their stimulation circuitries 28 and 58 to produce stimulation with a desired amplitude and timing described earlier. Both devices 60 and 70 may also be used to adjust one or more stimulation parameters of a stimulation program that the IPG 10 or ETS 50 is currently executing. Devices 60 and 70 may also wirelessly receive information from the IPG 10 or ETS 50, such as various status information, etc.

External controller 60 can be as described in U.S. Patent Application Publication 2015/0080982 for example, and may comprise a controller dedicated to work with the IPG 10 or ETS 50. External controller 60 may also comprise a general purpose mobile electronics device such as a mobile phone which has been programmed with a Medical Device Application (MDA) allowing it to work as a wireless controller for the IPG 10 or ETS 50, as described in U.S. Patent Application Publication 2015/0231402. External controller 60 includes a Graphical User Interface (GUI), preferably including means for entering commands (e.g., buttons or selectable graphical icons) and a display 62. The external controller 60′s GUI enables a patient to adjust stimulation parameters, although it may have limited functionality when compared to the more-powerful clinician programmer 70, described shortly.

The external controller 60 can have one or more antennas capable of communicating with the IPG 10 and ETS 50. For example, the external controller 60 can have a near-field magnetic-induction coil antenna 64a capable of wirelessly communicating with the coil antenna 27a or 56a in the IPG 10 or ETS 50. The external controller 60 can also have a far-field RF antenna 64b capable of wirelessly communicating with the RF antenna 27b or 56b in the IPG 10 or ETS 50.

Clinician programmer 70 is described further in U.S. Patent Application Publication 2015/0360038, and can comprise a computing device 72, such as a desktop, laptop, or notebook computer, a tablet, a mobile smart phone, a Personal Data Assistant (PDA)-type mobile computing device, etc. In FIG. 5, computing device 72 is shown as a laptop computer that includes typical computer user interface means such as a screen 74, a mouse, a keyboard, speakers, a stylus, a printer, etc., not all of which are shown for convenience. Also shown in FIG. 5 are accessory devices for the clinician programmer 70 that are usually specific to its operation as a stimulation controller, such as a communication “wand” 76 coupleable to suitable ports on the computing device 72, such as USB ports 79 for example.

The antenna used in the clinician programmer 70 to communicate with the IPG 10 or ETS 50 can depend on the type of antennas included in those devices. If the patient's IPG 10 or ETS 50 includes a coil antenna 27a or 56a, wand 76 can likewise include a coil antenna 80a to establish near-field magnetic-induction communications at small distances. In this instance, the wand 76 may be affixed in close proximity to the patient, such as by placing the wand 76 in a belt or holster wearable by the patient and proximate to the patient's IPG 10 or ETS 50. If the IPG 10 or ETS 50 includes an RF antenna 27b or 56b, the wand 76, the computing device 72, or both, can likewise include an RF antenna 80b to establish communication with the IPG 10 or ETS 50 at larger distances. The clinician programmer 70 can also communicate with other devices and networks, such as the Internet, either wirelessly or via a wired link provided at an Ethernet or network port.

To program stimulation programs or parameters for the IPG 10 or ETS 50, the clinician interfaces with a clinician programmer GUI 82 provided on the display 74 of the computing device 72. As one skilled in the art understands, the GUI 82 can be rendered by execution of clinician programmer software 84 stored in the computing device 72, which software may be stored in the device's non-volatile memory 86. Execution of the clinician programmer software 84 in the computing device 72 can be facilitated by control circuitry 88 such as one or more microprocessors, microcomputers, FPGAs, DSPs, other digital logic structures, etc., which are capable of executing programs in a computing device, and which may comprise their own memories. In one example, control circuitry 88 may comprise an i5 processor manufactured by Intel Corp., as described at https://www.intel.com/content/www/us/en/products/processors/core/i5-processors.html. Such control circuitry 88, in addition to executing the clinician programmer software 84 and rendering the GUI 82, can also enable communications via antennas 80a or 80b to communicate stimulation parameters chosen through the GUI 82 to the patient's IPG 10 or ETS 50.

The GUI of the external controller 60 may provide similar functionality because the external controller 60 can include the same hardware and software programming as the clinician programmer. For example, the external controller 60 includes control circuitry 66 similar to the control circuitry 88 in the clinician programmer 70, and may similarly be programmed with external controller software stored in device memory.

### SUMMARY

A method for providing stimulation to a patient is disclosed, which may comprise: providing a plurality of electrodes of a spinal cord stimulator proximate to a patient's spinal cord; selecting at least one electrode to recruit neural elements of the patient's spinal cord; and providing, from stimulation circuitry in the spinal cord stimulator, waveforms to the selected at least one electrode to cause stimulation of the patient's spinal cord, wherein the waveforms comprise a first phase of a first polarity during a first duration and a second phase of a second polarity opposite the first polarity during a second duration following the first duration, wherein the first duration is greater than 2.0 ms and less than 500 ms, and wherein the first phase lacks a quiescent period during which no stimulation is provided from the stimulation circuitry to the patient's spinal cord.

The first duration may be 2.6 ms or greater, and/or 10 ms or less. The second duration may be greater than 2.0 ms and less than 500 ms, and the second phase may lack a quiescent period during which no stimulation is provided from the stimulation circuitry to the patient's spinal cord. The second duration may be 2.6 ms or greater, and or 10 ms or less.

At least one of the first phase or the second phase may comprise concatenated first and second sub-phases, wherein one of the sub-phases may be actively driven with a current by the stimulation circuitry, and wherein the other of the sub-phases may be passively driven by the stimulation circuitry. The first and second phases may be actively driven with a current by the stimulation circuitry over their respective entireties.

The first and second phases may be or may not be charge balanced at each of the at least one electrodes for at least some of the waveforms. The waveforms may be provided to the selected at least one electrode at one or more frequencies comprising 60 Hz or less.

The at least one electrode may be selected to selectively recruit neural elements in a dorsal horn of the patient's spinal cord. The stimulation may comprise sub-perception stimulation. At least one of the first phase or the second phase may comprise a current amplitude of 0.6 mA or less over its respective entire duration.

The at least one electrode may be selected to selectively recruit neural elements in a dorsal column of the patient's spinal cord. The stimulation may comprises supra-perception stimulation. At least one of the first phase or the second phase may comprise a current amplitude of greater than 0.6 mA over its respective entire duration. The stimulation may comprises sub- perception stimulation but may be above a dorsal column activation threshold.

Stimulation parameters for the waveforms may be provided to the stimulation circuitry by a single timing channel circuitry of the spinal cord stimulator. The selected at least one electrode comprises one or more anodic electrodes and one or more cathodic electrodes. One of the selected at least one electrode comprises a case electrode.

A spinal cord stimulator for providing stimulation to a patient is disclosed, which may comprise: a plurality of electrodes each configured to be placed proximate to a patient's spinal cord; stimulation circuitry configurable to select at least one electrode to recruit neural elements in the patient's spinal cord; and wherein the stimulation circuitry is configured to provide a series of waveforms to the selected at least one electrode to cause stimulation in the patient's spinal cord, wherein the waveforms comprise a first phase of a first polarity during a first duration and a second phase of a second polarity opposite the first polarity during a second duration following the first duration, wherein the first duration is 2.6 ms or greater and less than 500 ms, and wherein the first phase lacks a quiescent period during which no stimulation is provided from the stimulation circuitry to the patient's spinal cord.

The first duration may be 10 ms or less. The second duration may be greater than 2.6 ms and less than 500 ms, and the second phase may lack a quiescent period during which no stimulation is provided from the stimulation circuitry to the patient's spinal cord. The second duration may be 10 ms or less.

The stimulation circuitry may comprise: one or more current sources configured to provide a current to the selected at least two of the electrodes; and passive recovery circuitry configured to couple the electrodes to a common potential.

At least one of the first phase or the second phase may comprise concatenated first and second sub-phases, wherein one of the sub-phases may be actively driven with a current by the one or more current sources, and wherein the other of the sub-phases may be passively driven by the passive recovery circuitry.

The first and second phases may be actively driven with a current by the one or more current sources over their respective entireties.

The first and second phases may be or may not be charge balanced at each of the at least one electrodes for at least some of the waveforms. The waveforms may be provided to the selected at least one electrode at one or more frequencies comprising 60 Hz or less.

The stimulation circuitry may be configurable to select the at least one electrode to selectively recruit neural elements in a dorsal horn of the patient's spinal cord or in a dorsal column of the patient's spinal cord.

The stimulation may comprise sub-perception stimulation. At least one of the first phase or the second phase may comprise a current amplitude of 0.6 mA or less over its respective entire duration. The stimulation may comprise supra-perception stimulation. At least one of the first phase or the second phase may comprise a current amplitude of greater than 0.6 mA over its respective entire duration.

The spinal cord stimulator may further comprise a plurality of timing channel circuitries configured to provide stimulation parameters to the stimulation circuitry, wherein stimulation parameters for the waveforms are provided by a single one of the timing channel circuitries. The selected at least one electrode may comprise one or more anodic electrodes and one or more cathodic electrodes. One of the selected at least one electrode comprises a case electrode.

A method for providing stimulation to a patient is disclosed, which may comprise: providing a plurality of electrodes of a spinal cord stimulator proximate to a patient's spinal cord; selecting at least one electrode to recruit neural elements of the patient's spinal cord; and providing, from stimulation circuitry in the spinal cord stimulator, waveforms to the selected at least one electrode to cause stimulation of the patient's spinal cord, wherein the waveforms comprise a first phase of a first polarity, and a second phase of a second polarity opposite the first polarity, wherein the first phase comprises a first sub-phase during a first duration and a second sub-phase during a second duration, the two sub-phases of the first polarity separated by the second phase during a third duration, and wherein a sum of the first and second durations is greater than 2.0 ms and less than 500 ms.

The second phase may lack a quiescent period during which no stimulation is provided from the stimulation circuitry to the patient's spinal cord.

At least one of the sub-phases may be actively driven with a current by the stimulation circuitry, and at least one of the other sub-phases may be passively driven by the stimulation circuitry.

The sub-phases may be actively driven with a current by the stimulation circuitry.

The third duration may be 2.0 ms or greater, and/or 10 ms or less.

At least one of the sub-phases may lack a quiescent period during which no stimulation is provided from the stimulation circuitry to the patient's spinal cord. The sum of the first and second durations may be 2.6 ms or greater, and/or 10 ms or less.

The second phase may comprise concatenated first and second sub-phases, wherein at least one of the sub-phases of the second phase may be actively driven with a current by the stimulation circuitry, and wherein at least one of the other sub-phases may be passively driven by the stimulation circuitry.

The sub-phases of the first polarity and the second phase of the second polarity may be actively driven with a current by the stimulation circuitry over their respective entireties.

The first and second phases may be or may not be charge balanced at each of the at least one electrodes for at least some of the waveforms. The waveforms may be provided to the selected at least one electrode at one or more frequencies comprising 60 Hz or less.

The at least one electrode may be selected to selectively recruit neural elements in a dorsal horn of the patient's spinal cord. The stimulation may comprise sub-perception stimulation. The second phase may comprise a current amplitude of 0.6 mA or less over its respective entire duration.

The at least one electrode may be selected to selectively recruit neural elements in a dorsal column of the patient's spinal cord. The stimulation may comprise supra-perception stimulation. The second phase may comprise a current amplitude of greater than 0.6 mA over its respective entire duration. The stimulation may comprise sub-perception stimulation but above a dorsal column activation threshold.

Stimulation parameters for the waveforms may be provided to the stimulation circuitry by a single timing channel circuitry of the spinal cord stimulator.

The selected at least one electrode may comprise one or more anodic electrodes and one or more cathodic electrodes. One of the at least one selected electrode comprises a case electrode.

A spinal cord stimulator for providing stimulation to a patient is disclosed, which may comprise: a plurality of electrodes each configured to be placed proximate to a patient's spinal cord; and stimulation circuitry configurable to select at least one electrode to recruit neural elements in a patient's spinal cord; wherein the stimulation circuitry is configured to provide a series of waveforms to the selected at least one electrode to cause stimulation in the patient's spinal cord, wherein the waveforms comprise a first phase of a first polarity, and a second phase of a second polarity opposite the first polarity, wherein the first phase comprises a first sub-phase during a first duration and a second sub-phase during a second duration, the two sub-phases of the first polarity separated by the second phase during a third duration, and wherein a sum of the first and second durations is 2.6 ms or greater and less than 500 ms.

The second phase may lack a quiescent period during which no stimulation is provided from the stimulation circuitry to the patient's spinal cord.

The third duration may be 2.0 ms or greater, and/or 10 ms or less.

At least one of the sub-phases may lack a quiescent period during which no stimulation is provided from the stimulation circuitry to the patient's spinal cord.

The sum of the first and second durations may be 10 ms or less.

The stimulation circuitry may comprise: one or more current sources configured to provide a current to the selected at least two of the electrodes; and passive recovery circuitry configured to couple the electrodes to a common potential.

The second phase may comprise concatenated first and second sub-phases, and at least one of the sub-phases of the second phase may be actively driven with a current by the one or more current sources, and at least one of the other sub-phases may be passively driven by the passive recovery circuitry.

The sub-phases of the first polarity and the second phase of the second polarity may be actively driven with a current by the one or more current sources over their respective entireties.

At least one of the sub-phases may be actively driven with a current by the one or more current sources, and at least one of the other sub-phases may be passively driven by the passive recovery circuitry.

The sub-phases may be actively driven with a current by the one or more current sources.

The first and second phases may be or may not be charge balanced at each of the at least one electrodes for at least some of the waveforms. The waveforms may be provided to the selected at least one electrode at one or more frequencies comprising 60 Hz or less.

The stimulation circuitry may be configurable to select the at least one electrode to selectively recruit neural elements in a dorsal horn of the patient's spinal cord or in a dorsal column of the patient's spinal cord. The stimulation may comprise sub-perception stimulation. At least one of the first phase or the second phase may comprise a current amplitude of 0.6 mA or less over its respective entire duration. The stimulation may comprise supra-perception stimulation. At least one of the first phase or the second phase may comprise a current amplitude of greater than 0.6 mA over its respective entire duration.

The spinal cord stimulator may further comprise a plurality of timing channel circuitries configured to provide stimulation parameters to the stimulation circuitry, wherein stimulation parameters for the waveforms are provided by a single one of the timing channel circuitries.

The selected at least one electrode may comprise one or more anodic electrodes and one or more cathodic electrodes. One of the at least one selected electrode may comprise a case electrode.

### BRIEF DESCRIPTION OF THE DRAWINGS

FIG. 1 shows an Implantable Pulse Generator (IPG), in accordance with the prior art.

FIGS. 2A and 2B show an example of stimulation waveforms producible by the IPG or in an External Trial Stimulator (ETS), in accordance with the prior art.

FIG. 3 shows stimulation circuitry useable in the IPG or ETS, in accordance with the prior art.

FIG. 4 shows an ETS environment useable to provide stimulation before implantation of an IPG, in accordance with the prior art.

FIG. 5 shows various external devices capable of communicating with and programming stimulation in an IPG and ETS, in accordance with the prior art.

FIGS. 6A and 6B show simulation of certain waveforms and assesses the firing rate of inhibitory interneurons IIN as are typically present in the dorsal horn of the spinal cord, and shows a simulated relationship between long phase duration and firing rate.

FIGS. 7A and 7B shows another simulation of a long duration phase, and shows how it can cause asynchronous firing of axons in the dorsal column.

FIG. 8 shows examples of triphasic and biphasic waveforms similar to those simulated, and how these waveforms can be formed in accordance with a specific IPG or ETS current generation architecture.

FIGS. 9A-9C show the specific architecture useable to form the waveforms of FIG. 8.

FIGS. 10A-10E show further examples of triphasic and biphasic waveforms similar to those simulated and having waveforms with phases of long durations which can be formed using generic IPG or ETS architectures.

FIG. 11 shows an example of how the waveforms with phases of long durations can be provided to one or more anode electrode and one or more cathode electrodes as useful to field shaping and current steering in a patient's tissue.

FIG. 12 shows that the waveforms with phases of long durations can be charge imbalanced for useful reasons.

FIG. 13 shows various examples of waveforms with phases of long durations.

FIG. 14 shows an example of how the waveforms with phases of long durations can be formed by mirroring a biphasic waveform around an axis.

FIGS. 15A and 15B show possible variations to the amplitudes and phase durations of the waveforms with phases of long durations.

FIG. 16 shows how the various examples of waveforms with phases of long durations can be mixed and matched to form various patterns or random patterns.

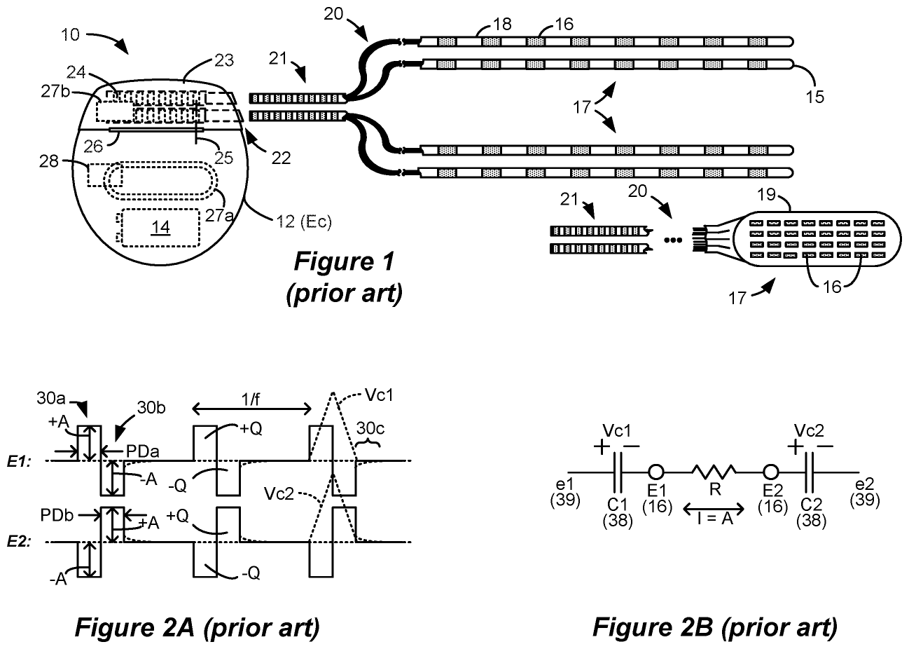

### DETAILED DESCRIPTION

While Spinal Cord Stimulation (SCS) therapy can be an effective means of alleviating a patient's pain, such stimulation can also cause paresthesia. Paresthesia—sometimes referred to a “supra-perception” therapy—is a sensation such as tingling, prickling, etc., that can accompany SCS therapy. Generally, the effects of paresthesia are mild, or at least are not overly concerning to a patient. Moreover, paresthesia can be a reasonable tradeoff for a patient whose chronic pain has now been brought under control by SCS therapy. Some patients even find paresthesia comfortable and soothing.

Nonetheless, at least for some patients, SCS therapy would ideally provide pain relief without sensations such as paresthesia—what is often referred to as “sub-perception” or sub-threshold therapy that a patient cannot feel. Effective sub-perception therapy may provide pain relief without paresthesia by issuing properly dosed stimulation waveforms at higher frequencies. Unfortunately, such higher-frequency stimulation may require more power, which tends to drain the battery 14 of the IPG or ETS. See, e.g., U.S. Patent Application Publication 2016/0367822. If an IPG's battery 14 is a primary cell and not rechargeable, high-frequency stimulation means that the IPG 10 will need to be replaced more quickly. Alternatively, if an IPG or ETS battery is rechargeable, the IPG 10 will need to be charged more frequently, or for longer periods of time. Either way, the patient can be inconvenienced.

The inventors have investigated stimulation waveforms which may be helpful in providing therapeutic relief for SCS patients, which therapy is preferably (but not necessarily) sub-perception in nature, and which further preferably occurs at lower frequencies more considerate of power draw. Of particular interest is an investigation into the role of inhibitory interneuron (IIN) cells and the terminals from descending pain inhibitory pathways present in the grey matter of the spinal cord, and in particular present in the dorsal horn of the spinal cord. It is theorized that electrical stimulation of these types of nerve cells release neurotransmitters that block pain signals from traveling in the spinal cord. In short, by stimulating IIN cells in a patient's spinal cord, pain signals may be blocked, thus providing pain suppression for the patient. Stimulation may also modulate IIN or other neural targets even if such neural targets are not activated (i.e., without causing action potentials), thus changing the excitability of such neural targets.

The inventors have simulated lower-frequency (e.g., 500 Hz or less) waveforms to understand their effect on IIN cells. Simulation involved using finite element models (FEMs) using published geometric and electrical tissue properties and clinically utilized SCS electrode geometries. FEM-derived extracellular potentials were coupled to biophysical models of IIN cells to simulate the neurophysiological effects for waveforms of different shapes and frequencies. For each waveform simulated, axon activation thresholds were quantified and compared to determine a firing rate of IIN cells in the spinal cord.

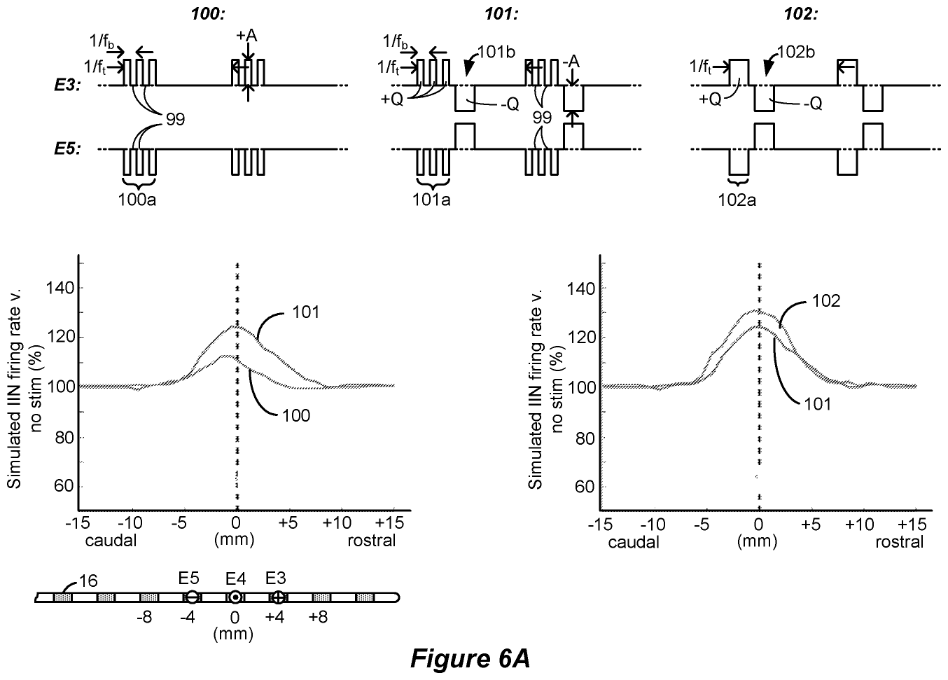

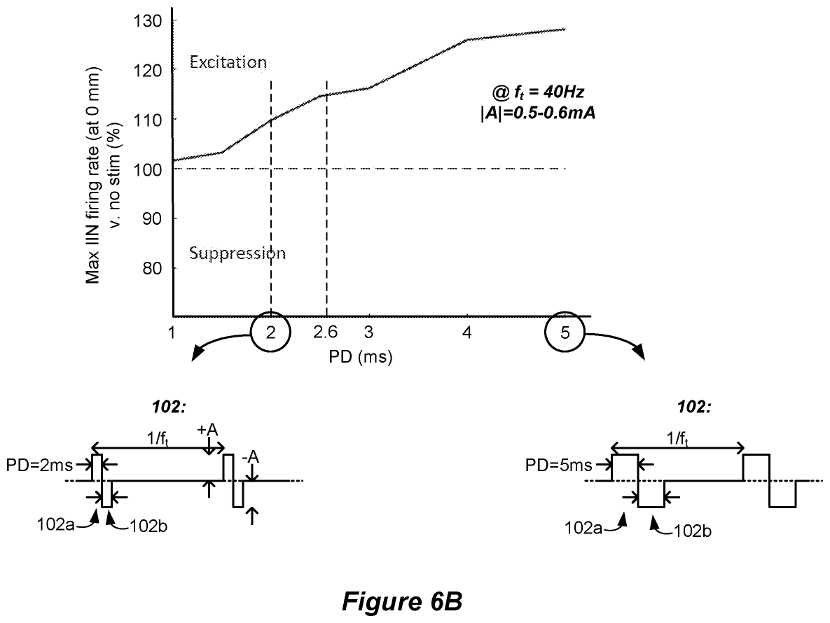

Simulation results are shown in FIG. 6A for waveforms 100, 101, and 102. Generally speaking, the waveforms comprise phases, described below, which have phase durations PD that are longer (e.g., greater than 2.0 ms) and amplitudes A that are lower (e.g., less than or equal to 1 mA) than those typically used to provide SCS therapy. Such waveforms were interesting to investigate because TIN cells in the dorsal horn are believed to continuously fire (and thus provide pain suppression) during the provision of long duration phases of current. Excitatory neurons in the dorsal horn, by contrast, can exhibit adaptive behavior, such that they will fire at beginning of a waveform phase, but can stop even if the current to the tissue is continued. Further, simulation of longer phase duration, lower amplitude waveforms is thought to be below the rheobase of excitatory neurons.

Sub-perception amplitudes were chosen and simulated by assessing a threshold at which dorsal column axons were stimulated. In this regard, it is known that stimulation of dorsal column axons can cause paresthesia. Therefore, the simulation also involved determining an amplitude threshold at which dorsal column axons would be recruited (i.e., fire) for given stimulation parameters (e.g., PD, f, etc.). Once this paresthesia amplitude threshold was determined, the amplitude of the waveforms was set to 90% of the paresthesia amplitude threshold, thus allowing the simulation of sub-perception (paresthesia free) stimulation. Although the sub-perception amplitude A (+A or −A) varied for the different waveforms, it was generally in the range of 0.45 to 0.6 mA, thus suggesting that current amplitudes of 0.6 mA or lower would provide sub-perception stimulation for the stimulation parameters chosen, in particular the phase durations.

During the simulation, bipolar stimulation was used with electrode E3 comprising an anode electrode and electrode E5 comprising a cathode electrode (during first phases 100a, 101a, and 102a), as shown in the lead at the bottom of FIG. 6A. In the simulation, it was assumed that the electrodes 16 were spaced at 4 mm in the rostro-caudal (head-feet) direction, and thus stimulation at electrodes E3 and E5 created a bipole with a 8 mm spacing centered at electrode E4.

Each of the simulated waveforms 100, 101 and 102 have different attributes theorized as possibly relevant to the rate at which inhibitory interneurons might fire in the spinal cord. For example, waveforms 100 comprise a burst of a few monophasic pulses of a single polarity which occur during a phase 100a. These monophasic pulses as simulated issue at a burst frequency f_{b} on the order of 200 to 500 Hz, while the waveforms 100 were issued at a tonic frequency fₜ on the order of 20 to 60 Hz. The monophasic pulses during phase 100a were simulated to have quiescent periods 99 between them during which no stimulation (e.g., no current or charge) was provided to the tissue. Although not simulated, it would be expected that waveforms 100 if actually implemented in a patient might be followed by a passive charge recovery period (e.g., 30c, as explained earlier).

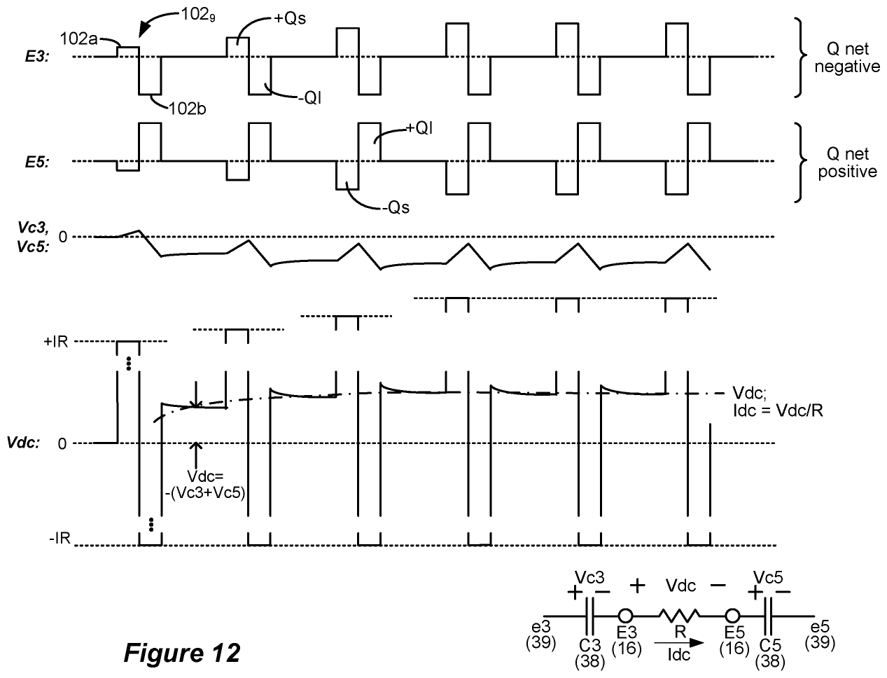

For comparative purposes, waveforms 101 were also simulated. Waveforms 101 are similar to waveforms 100, and include a phase 101a again having a burst of monophasic pulses with quiescent periods 99 between the monophasic pulses. Waveforms 101 however differ in that a single rectangular active charge recovery phase was also simulated. This occurs during phase 101b, during which a current of an opposite polarity (and again with an amplitude on the order of 0.45 to 0.6 mA) was simulated to recover charge stored on capacitive elements (e.g., C3 and C5) in the current path. To simulate perfect charge recovery at each electrode, the charge of the monophasic pulses during phase 101a (e.g., +Q at E3) was made equal and opposite to the charge of the active charge recovery pulse during phase 101b (e.g., −Q at E3). However, as discussed further below with reference to FIG. 12, stimulation waveforms may not always be charge balanced, and there can be advantages to using charge imbalanced waveforms.

The simulated inhibitory interneuron (IIN) firing rate for simulated waveforms 100 and 101 is shown in the left graph of FIG. 6A. The figure shows a simulated percentage increase in TIN firing rate compared to when no situation is provided (100%), as measured from the center (e.g., E4, 0 mm) of the bipole formed at E3/E5. As would be expected, the IIN firing rate is maximized at E4 where the electric field in the tissue would be the strongest, and falls off back to a non-stimulation baseline (100%) more or less at the location of the electrodes (e.g., E3 and E5) chosen for stimulation. The simulated IIN firing rate was higher for waveforms 101 than for waveforms 100. This suggests that, in some examples, the use of rectangular active charge recovery (101b) may be beneficial, possibly because a constant current amplitude is present for the entire duration of the active charge recovery phase 101b.

From this graph it was hypothesized that what might be noteworthy for increasing the TIN firing rate, and thus increasing pain suppression in SCS, is the unusually-long phase durations (e.g., greater than 2.0 ms) of the active charge recovery phase 101b, and that the use of preceding monophasic burst pulses is different from what was previously believed. In this regard, waveforms 102 were also simulated which kept an active charge recovery phase 102b (similar to 101b), but which was preceded by a similarly long continuous first phase 102a lacking in burst pulses. That is, neither phases 102a nor 102b had quiescent periods 99, but instead provided current or charge to the tissue throughout their durations. The charge of the single phase 102a (e.g, +Q at E3) was again made equal and opposite to the charge of the active current recovery phase 102b (e.g., −Q at E3) to simulate perfect charge recovery. As shown in the right graph, the IlN firing rate was even higher for waveforms 102 than for waveforms 101. In short, waveforms 102 were noticed during simulation to be superior to waveforms 100 and 101 in regards to TIN firing rate.

From these simulated observations, the inventors hypothesize that, in some circumstances, the provision of long phase duration, low amplitude waveforms to the patient may be support enhanced IIN excitability. Such a result is encouraging to observe, because it suggests that effective sub-perception therapy can be provided without the necessity and complication of providing burst pulses at higher frequencies (f_{b}) that may be less considerate of IPG power. Instead, effective results are shown using waveforms 102 issued at lower tonic frequency fₜ.

FIG. 6B shows further simulation of waveforms 102, and in particular shows the effect of variation of the phase duration PD of each of the phases 102a and 102b on the IIN firing rate. Specifically, the graph shows the maximum IlN firing rate experienced in the middle of the simulated bipole (at 0 mm, E4) at phase durations ranging from 1 to 5 ms. At each simulated phase duration, the tonic frequency fₜ was kept constant at 40 Hz. The phases 102a and 102b were symmetric, having the same amplitude and phase duration, and again no quiescent periods 99. For each tested phase duration, the amplitude was adjusted to 90% of the paresthesia amplitude threshold to create sub-perception waveforms which would not recruit dorsal column axons, as discussed above. Thus, the amplitude of each waveform was adjusted as necessary, and varied in a range of 0.45 to 0.6 mA for the waveforms simulated in FIG. 6B. The graph shows that the IIN firing rate increased with increasing phase duration PD despite the stimulation amplitude being sub-perception, which suggests that longer phase durations may be more effective in suppressing pain in SCS patients. Increased TIN firing may be noteworthy (>110%) at phase durations of greater than 2.0 ms, and even further noteworthy (>115%) at phase durations of 2.6 ms or greater.

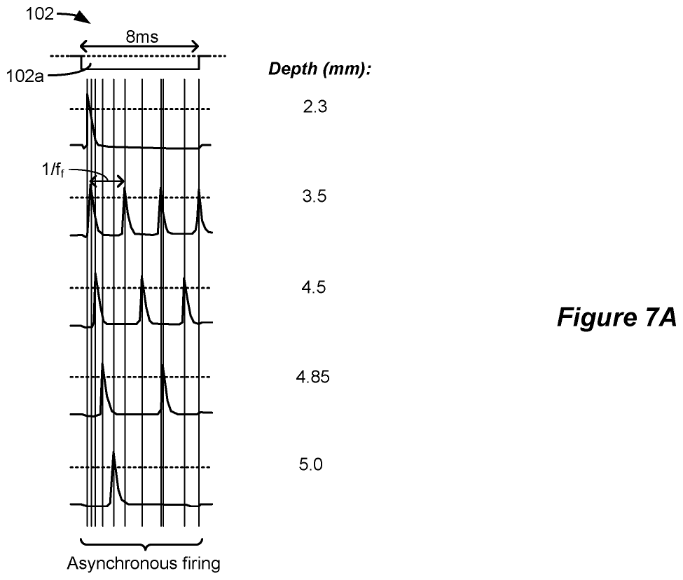

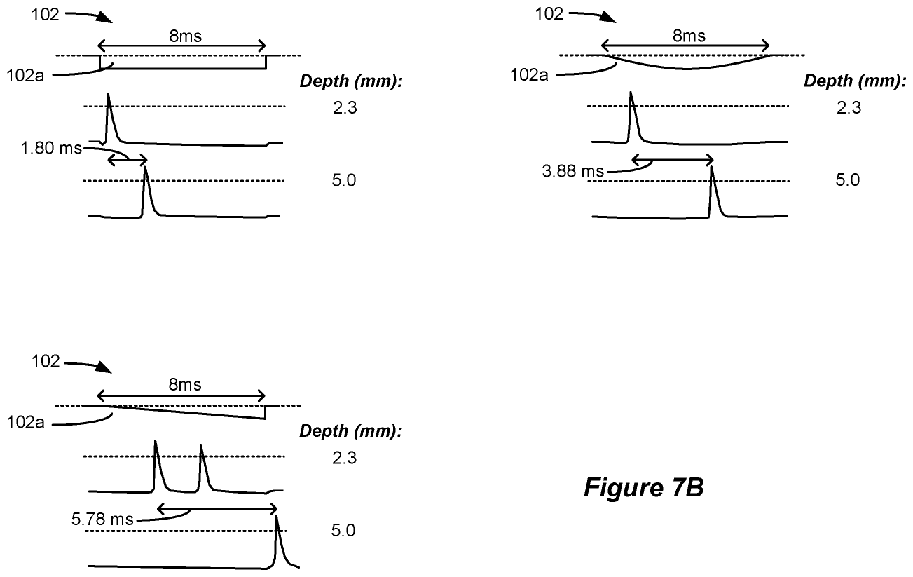

The use of long phase durations in SCS stimulation can have benefits beyond the recruitment of TIN cells. There can also be value in stimulating dorsal column axons, as occurs in traditional SCS therapies, which may be supra-perception. FIG. 7A shows another simulation that is useful in understanding the effect that long phase durations can have on dorsal column axons. Shown in particular is a waveform 102, which as simulated has a phase 102a that is 8 ms in duration. Although not shown, it should be understood that the waveform 102 could have another phase (102b) useful for active or passive charge recovery, and that could also be of long duration for therapeutic effectiveness. Only the phase 102a at a single electrode is shown, but again in an actual implementation a return electrode would also be chosen.

The simulation shows action potentials generated in the dorsal column axons at different depths in the patient's tissue. As can be seen, the axons fire (depolarize) at different frequencies (f_{f}) as a function of their depth in the tissue from the electrodes chosen to provide the stimulation. Closer to the surface (e.g., at 2.3 mm), a single action potential issues near to the beginning of the 8 ms phase 102a. At 3.5 mm, four action potentials issue during the 8 ms phase duration, which comprised the maximum firing frequency f_{f} noticed. At lower depths, the firing frequency declined to three (4.5 mm), two (4.85 mm), and one (5 mm) action potentials per the 8 ms phase duration.

Long phase duration pulses of different shapes can also stimulate dorsal column axons at different depths and with different frequencies, as shown in the simulations of FIG. 7B. The top left shows simulation of a square pulse having a constant current amplitude. In this example, dorsal column axons are again stimulated differently at different depths, with shallower axons (2.3 mm) firing first, and relatively quickly after the beginning of the pulse. Deeper axons (5.0 mm) fire relatively quickly after the shallower pulse (within 1.80 ms). The top right shows simulation of a half sinusoid pulse. Again, dorsal column axons are stimulated differently at different depths, with shallower axons (2.3 mm) firing first but later after the beginning of the pulse, and deeper axons (5.0 mm) firing significantly later after the shallower pulse (within 3.88 ms). The bottom left shows simulation of a linear ramp pulse. In this example, shallower axons (2.3 mm) fire first but significantly later after the beginning of the pulse, and deeper axons (5.0 mm) fire even more significantly later after the shallower pulse (within 5.78 ms).

In certain circumstances, notably, and as best seen in FIG. 7A, firing of the axons at the different depths may be asynchronous, as governed by the different firing frequencies noticed at each depth, and different delays in firing after the beginning of the phase 102a. Asynchronous firing can reduce or eliminate the effects of paresthesia. See, e.g., U.S. Patent Application Publication 2017/0296823, which is incorporated herein by reference. In other words, stimulation may be provided that is sub-perception, but above the dorsal column activation threshold, such that dorsal column axons fire action potentials but those action potentials are not substantially felt by the patient, e.g., in the form of paresthesia or some other sensation.

It is theorized that the observed results may be associated with the persistent sodium current component of the membrane dynamic model, and the closing of the h-gates in the sodium channels. Axons very close to the electrode will remain depolarized during the long duration of the phase, and the h-gates will remain shut. Axons farther away will receive a lower strength depolarizing effect, and as a result the h-gates will not close as much compared to the axons near the electrode, allowing for the re-firing of axons due to the presence of the persistent sodium current.

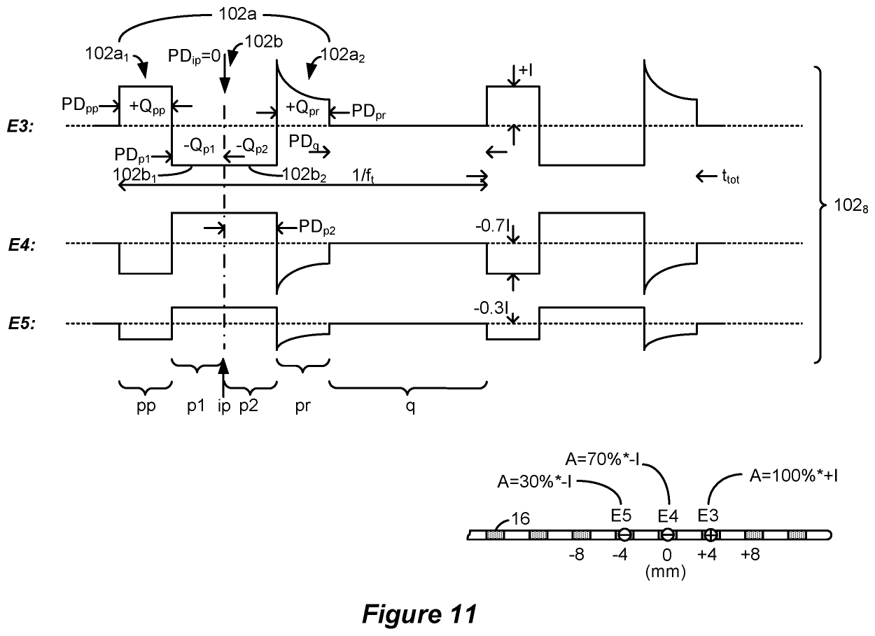

Using long phase durations, it is therefore possible to place the electrode near or in contact with a target structure to provide stronger response (more action potentials) in nerves further from the electrodes. By contrast, when conventional shorter phase durations are used in an SCS application, axons near the electrodes will be stimulated more than axons further away from the electrodes. As a result, use of longer phase durations does not require rigorous precision in locating a source of pain in the dorsal column, and electrode selection is therefore theorized to be less demanding. Nonetheless, electrode selection and current steering can also be used, as described later with reference to FIG. 11.

Additionally, selectivity of axons can be obtained based on axon diameters. Large diameter axons are usually recruited at lower thresholds than small diameter axons. Because the activating function of smaller diameter fibers is often smaller than that of large diameter fibers, small diameter fibers often behave similar to larger diameter fibers further away from the electrode (in terms of stimulation threshold response). By using large phase durations during stimulation it is therefore possible to depolarize large diameter fibers and shut the h-gates, while keeping h-gates more open in smaller diameter fibers and allowing for those fibers to refire. This will allow reversing the order of recruitment of axon fibers by diameter and allow smaller fibers to be stimulated with a higher firing frequency f_{f} than larger fibers.

Note that the simulations of FIGS. 6A-6B (assessing IIN cells in the dorsal horn), and FIG. 7 (assessing dorsal column axons), are not mutually exclusive. That is, a long phase duration can both excite IIN cells, and excite dorsal column axons in an asynchronous manner, both of which can provide pain suppression whether accompanied by paresthesia or not. In fact, a single long phase duration could excite different neural targets differently, with some targets providing sub-perception therapy (e.g., IIN cells) and others providing supra-perception therapy (e.g., dorsal column axons).

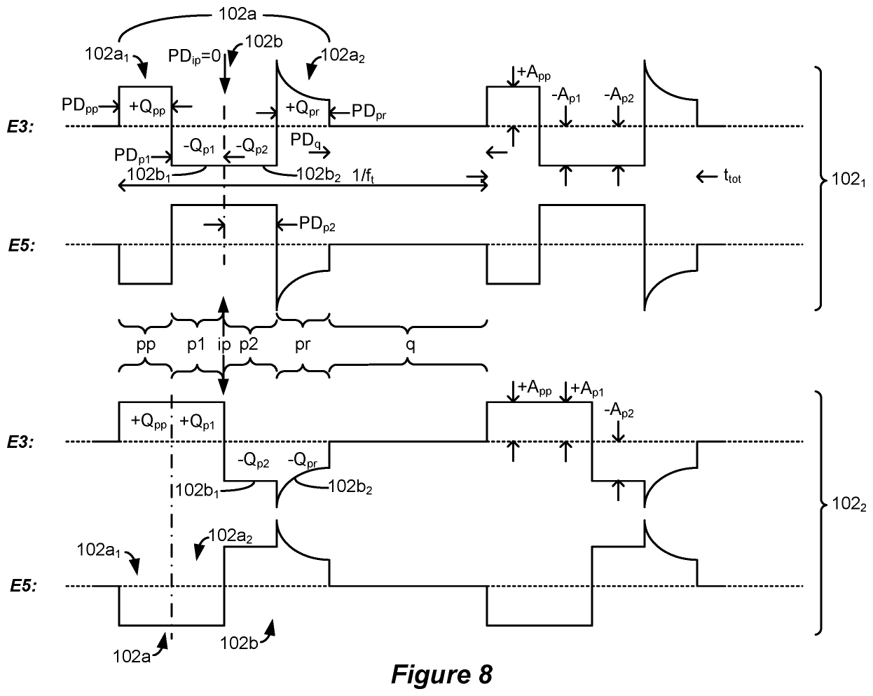

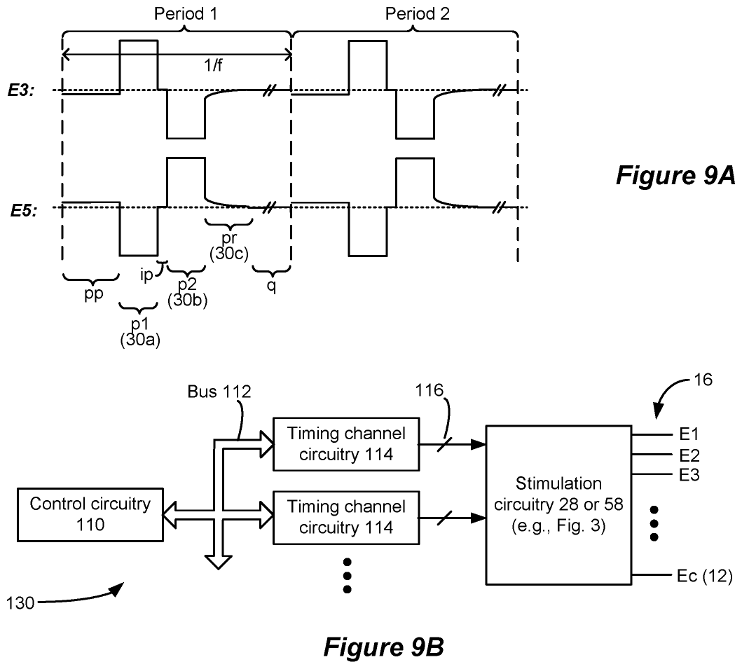

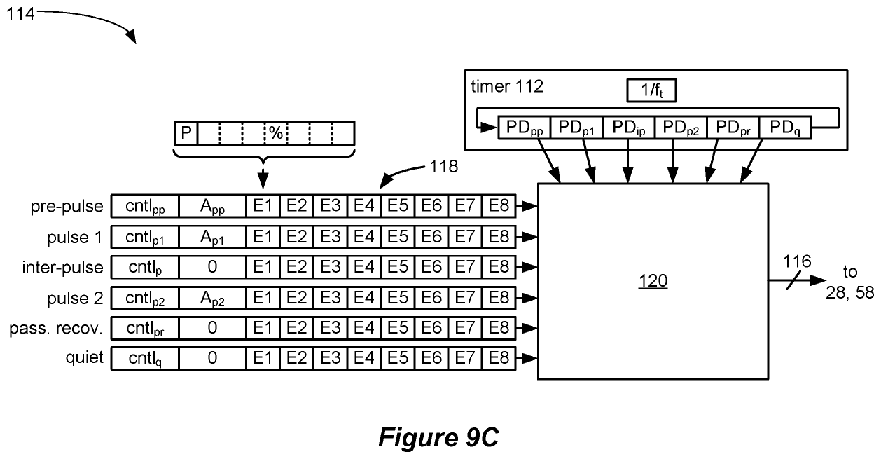

FIG. 8 shows two examples by which the waveforms 102 can be formed in an IPG or ETS. In FIG. 8, the waveforms 102 have phases 102a and 102b that are defined in six phases in accordance with a specific, non-limiting IPG or ETS current generation architecture 130 that will be explained further with respect to FIGS. 9A-9C. Specifically, the phases 102a and 102b are defined using a pre-pulse phase ‘pp’, a first active phase ‘p1’, an interpulse phase ‘ip’, a second active phase ‘p2’, a passive recovery phase ‘pr’, and a quiet phase ‘q’, consistent with the phases that the architecture 130 supports. As discussed further below, waveforms 102 can be formed in a single timing channel of the IPG or ETS which simplifies programming. One skilled will understand that first phase p1 is analogous to first phase 30a, second phase p2 is analogous to the second phase 30b, and passive recovery phase pr is analogous to the passive recovery phase 30c, all described earlier (FIG. 2A). Neither of phases 102a and 102b have quiescent periods during which no stimulation is provided from the stimulation circuitry to the patient, and instead provide current or charge to the tissue throughout their durations.

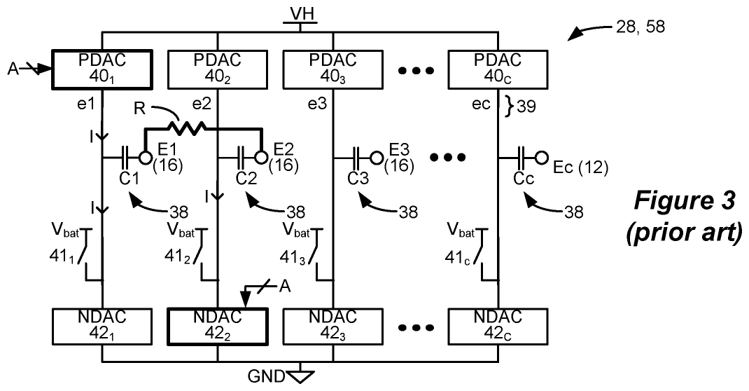

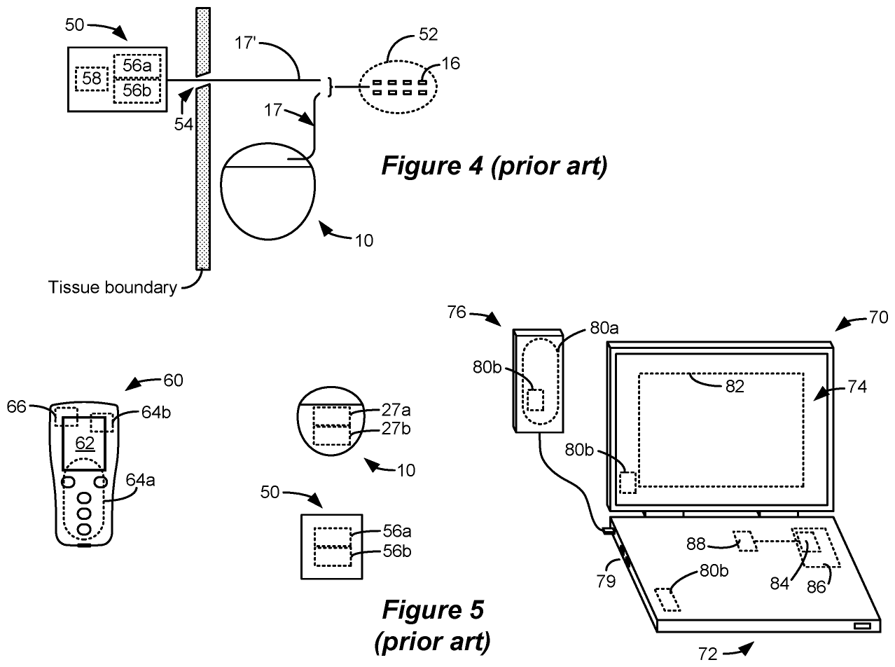

The pre-pulse phase pp, first phase p1, and second phase p2 comprise current amplitudes that are actively driven by the stimulation circuitry 28 or 58 (e.g., by one or more current sources such as PDAC(s) 40ᵢ and NDAC(s) 42ᵢ, FIG. 3) in the IPG or ETS. As explained further below, the pre-pulse phase pp is used differently in forming waveforms 102 from the manner in which this phase is typically used, in the sense that this phase is driven with a current amplitude that is higher than usual, and is on the order of, or equals, the current amplitude used during the first and second phases p1 and p2. The polarity of the pre-pulse phase pp may also be the same polarity as the first phase p1, which is also atypical. The first and second phases p1 and p2 may also be of the same polarity, which again is atypical, because these two phases are usually used to provide a first phase (30a) and second active charge recovery phase (30b) of an opposite polarity.

The interpulse phase ip, passive recovery phase pr, and quiet phase q are not phases that are actively driven with a current by the stimulation circuitry 28 or 58. The interpulse phase ip typically provides a short duration between the first and second phases p1 and p2 during which no current is provided to the tissue. The interpulse phase can be used to allow the nerves being stimulated to stabilize between the first and second phases p1 and p2, and to allow the stimulation circuitry 28 or 58 in the IPG or ETS time to set the circuitry as necessary to (typically) reverse the polarity of the current between these phases. The passive recovery phase pr as before (30c) prescribes a time after the active phases are driven (pp, p1, and p2) during which passive recovery of charge can occur through the closure of one or more passive recovery switches 41ᵢ (see FIG. 3). The quiet phase q follows the passive recovery phase pr, and essentially acts as a waiting phase before the next period of phases issues (starting with the pre-pulse phase pp of the next waveform). The duration of the quiet phase q is also used to set the tonic frequency fₜ at which the waveforms 102 will issue.

FIG. 8 shows two examples of waveforms 102 which can be used in an IPG or ETS to provide stimulation to a patient's tissue, which waveforms 102 are consistent with the waveforms learned to be beneficial during the simulations of FIGS. 6A and 6B and FIG. 7. The waveforms are shown as provided to electrodes E3 and E5 as before, but of course in an actual implementation the waveforms could be provided to any two or more electrodes (including the case electrode Ec 12) deemed suitable to treat a patient's pain, as discussed further below with reference to FIG. 11.

In the examples that follow, the waveforms 102 have two phases 102a and 102b of opposite polarities at each of the electrodes, similar to the waveforms simulated earlier. However, either or both of phases 102a and 102b may be broken down into sub-phases of the same polarity.

The first example 102₁ shows waveforms that are effectively triphasic in nature, which occurs by separating phase 102a into two sub-phase 102a₁ and 102a₂ of a first polarity, and separated by phase 102b of a separate polarity. Waveforms 102₁ of this type were also simulated and shown to have good effect on the TIN firing rate, although the simulation results for waveforms 102₁ was not summarized earlier. Triphasic waveforms may be preferable in an actual implementation, because it can reduce the magnitude of charge that might build on structures in the current path between the electrodes, and hence reduce voltages that may cause electrochemical reactions at the electrode/tissue interface.

In this example, sub-phase 102a₁ of phase 102a is formed during the pre-pulse phase pp supported by the architecture 130, and has a phase duration PDₚₚ and an amplitude of +Aₚₚ (at electrode E3, or −Aₚₚ at return electrode E5). Phase duration PDₚₚ may be a maximum that architecture 130 will allow. Amplitude Aₚₚ preferably comprises a value of 0.6 mA or lower, as this was shown during simulation to provide paresthesia-free, sub-perception therapy. However, it is not strictly necessary that waveforms 102 provide sub-perception therapy, and thus the amplitudes can be higher if desired. Sub-phase 102a₁ provides a total charge of +Qₚₚ=+Aₚₚ*PDₚₚ (at electrode E3, or −Qₚₚ at return electrode E5).

Phase 102b in this example comprises two concatenated sub-phases 102b₁ and 102b₂, which correspond to the first and second phases p1 and p2 supported by the architecture 130. In this example, the phase duration PDᵢₚ of the interphase period ip between phases p1 and p2 is set to zero, thus allowing phase 102b to be established by the concatenated sub-phases 102b₁ and 102b₂ without any gaps. In this example, the current amplitudes during sub-phases 102b₁ and 102b₂ are −Aₚ₁ and −Aₚ₂ respectively (at electrode E3, or +Aₚ₁ and +Aₚ₂ at return electrode E5), and in the illustrated example these current amplitudes are equal, although they could also differ. Phase durations PDₚ₁ and PDₚ₂ may again be a maximum the current generation architecture 130 will allow. A phase (e.g., 102b) with a relatively long phase duration (e.g., of 2.0 ms or longer) can be formed using architecture 130 (e.g., by summing together phases p1 and p2). Phase 102b provides a total charge injection to the tissue of −Qₚ₁+−Qₚ₂ (at electrode E3, or Qₚ₁+Qₚ₂ at return electrode E5), where Qₚ₁=Aₚ₁*PDₚ₁ and Qₚ₂=Aₚ₂*PDₚ₂.

Sub-phase 102a₂ is established using the passive recovery phase pr of architecture 130, and unlike sub-phase 102a₁ and phase 102b, Sub-phase 102a₂ is not actively driven by the stimulation circuitry 28 or 58 in the IPG or ETS. Its amplitude is passively established by passive charge recovery circuitry (e.g., switches 41ᵢ, FIG. 3) and results from charge imbalance resulting at each electrode after the completion of phase 102b. Assume that |Qₚₚ|<|Qₚ₁|+|Qₚ₂|. This means that a charge of −Qₚᵣ|=|Qₚ₁+|Qₚ₂|−|Qₚₚ| will be remaining on capacitances in the current path between active electrode E3 and E5 at the end of phase 102b, most notably on DC-blocking capacitors C3 and C5 (see FIG. 3). When passive recovery switches 41, (FIG. 3) are closed during phase 102a₂, and as explained earlier, such capacitances in the current path will be placed in parallel between a reference voltage (e.g., Vbat) and the patient's tissue, causing an exponentially-decaying current to flow from E3 to E5, as shown in FIG. 8. Given the charge imbalance, sub-phase 102a₂ is of the same polarity as sub-phase 102a₂. The phase duration of sub-phase 102a₂, PDₚᵣ (i.e., the time during which passive recovery switches 41, are closed) may be programmable in the architecture 130 of the IPG or ETS. In one example, the duration PDₚᵣ of sub-phase 102a₂ may be long enough to passively recovery all charge stored on capacitances in the current path between the selected electrodes, meaning that the current between those electrodes would equal zero at the end of sub-phase 102a₂ (although this isn't shown in FIG. 8). Should this occur, the waveforms at each electrode are said to be charge balanced, in the sense that the charge stored on capacitances in the current path at the end of waveform 102, is zero before a next waveform is issued (i.e., before a next pre-pulse phase pp is issued). If the current does not equal zero at the end of sub-phase 102a₂, there is still a residual amount of charge remaining on capacitances in the current path, and thus waveforms at each electrode are not charge balanced. This can also be beneficial, as discussed below with reference to FIG. 12.

When phases 102a and 102b are considered together, the total duration of waveforms 102₁, tₜₒₜ, will equal the sum of PDₚₚ, PDₚ₁, PDₚ₂, and PDₚᵣ, and so may in one example comprise a value of 10.2 ms or less. Further, at least one phase 102b may be made greater than 2.0 ms, and even 2.6 ms or greater, which was shown by simulation to be effective, and which may be a duration longer than a pre-define phase that the IPG or ETS will support. Phase 102a in total (the combined durations of 102a, and 102a₂) may also be made greater than 2.0 ms, and even 2.6 ms or greater.

The second example in FIG. 8 shows waveforms 102₂ again having phases 102a and 102b of the opposite polarities. Unlike waveforms 102₁, waveforms 102₂ are effectively biphasic, as they comprise a full phase 102a of one polarity followed by a full phase 102b of another polarity. This is true even though each of phases 102a and 102b is divided into concatenated sub-phases. For example, phase 102a comprises concatenated sub-phases 102a, and 102a₂, which correspond to phases pp and p1 supported by the architecture 130 of the IPG or ETS. Because architecture 130 does not support a phase between pp and p1, phase 102a is established without any gaps between the sub-phases 102a₁ and 102a₂. In this example, the current amplitude during phases 102a₁ and 102a₂ are +Aₚₚ and +Aₚ₁ respectively (at E3), and in the illustrated example these current amplitudes are equal, although they could also differ. Phase durations PDₚₚ and PDₚ₁ may again be limited by the architecture 130. In some examples, the combined duration of phase 102a may comprise an atypically long duration for SCS applications. Phase 102a provides a total charge of Qₚₚ+Qₚ₁ (at electrode E3, or −Qₚₚ−Qₚ₁ at return electrode E5), where Qₚₚ=Aₚₚ*PDₚₚ and Qₚ₁=Aₚ₁*PDₚ₁.

Phase 102b in this example comprises concatenated sub-phases 102b₁ and 102b₂, which correspond to phases p2 and pr supported by the architecture 130 of the IPG or ETS. In this example, the phase duration PDᵢₚ of the interphase period ip between phases p1 and p2 (and between phases 102a and 102b) is set to zero, although this is not strictly necessary. In this example, the current amplitude during phase 2 is −Aₚ₂ (at E3). Phase p2 provides a total charge of −Qₚ₂ (at electrode E3, or +Qₚ₂ at return electrode E5), where Qₚ₂=Aₚ₂*PDₚ₂.

Phase pr is again not actively driven by the stimulation circuitry 28 or 58 in the IPG or ETS, and so its amplitude is passive and results from charge imbalance resulting at each electrode. Assume that |Qₚₚ|+|Qₚ₁|>|Qₚ₂| at electrode E3. This means that a charge of |Qₚᵣ|=|Qₚₚ|+|Qₚ₁|−|Qₚ₂| will be remaining on capacitances at the end of sub-phase 102b₁. When passive recovery switches 41ᵢ (FIG. 3) are closed during sub-phase 102b₂, and as explained earlier, an exponentially-decaying current will flow from E5 to E3, as shown in FIG. 8, which current is of the same polarity as issued during sub-phase 102b₁, thus establishing phase 102b as a phase with a common polarity. As before, the duration PDₚᵣ may be long enough to passively recovery all charge stored on capacitances in the current path between the selected electrodes, resulting in waveforms at each electrode that are charge balanced. However, the waveforms 102₂ at each electrode may also not be charge balanced (see FIG. 12).

Effectively, the waveforms 102₂ are biphasic, with phase 102a (concatenated sub-phases 102a₁ and 102a₂) comprising a phase of a first polarity, and phase 102b (concatenated sub-phases 102b₁ and 102b₂) comprising a phase of the opposite polarity, even though the current provided during phase 102b is generated using both active and passive techniques. When phases 102a and 102b are considered together, the total phase duration of the waveforms 102₂, tₜₒₜ, may equal the sum of PDₚₚ, PDₚ₁, PDₚ₂, and PDₚᵣ, and so may in one example comprise a value of 10.2 ms or less. Again, at least one phase 102b may be made greater than 2.0 ms, and even 2.6 ms or greater, and phase 102a in total may also be made greater than 2.0 ms, and even 2.6 ms or greater.

FIGS. 9A-9C show an architecture 130 employable in an IPG or ETS that can be used to form waveforms 102, which architecture is further described in U.S. Pat. No. 9,008,790. FIG. 9A shows typical waveforms formed in an IPG or ETS using architecture 130, which essentially comprise biphasic current pulses that issue currents of relatively high amplitudes and of opposite polarities during phases 1 and 2, akin to the waveforms of FIG. 2A. By comparison to waveforms 102₁ and 102₂ of FIG. 8, the pre-pulse phase pp typically has a relatively smaller amplitude than the currents issued during phases 1 and 2 (e.g., perhaps 10% or less), and is thought to be useful to assist in recruiting deeper nerves in an SCS application. Note that a pre-pulse phase pp if used is typically of opposite polarity to the phase p1 that follows (compare waveforms 102₂). Typical waveforms formed using architecture 130 and as shown in FIG. 9A may also employ an interpulse phase p1, a passive recovery phase pr, and a quiet phase q, as previously explained.

Architecture 130 for creating the waveforms 102 is shown in FIG. 9B and comprises timing channel circuitries 114 capable of providing stimulation parameters to control stimulation circuitry 28 or 58 (FIG. 3) via control signals 116. The timing channel circuitries 114 in turn may be controlled by control circuitry 110 via a bus 112. The controller circuitry 110 may comprise a microcontroller, such as Part Number MSP430, manufactured by Texas Instruments, which is described in data sheets at http://www.ti.com/1sds/ti/microcontroller/16-bit_msp430/overview.page? DCMP=MCU_other& HQS=msp430. The control circuitry 110 more generally can comprise a microprocessor, Field Programmable Grid Array, Programmable Logic Device, Digital Signal Processor or like devices. Control circuitry 110 may include a central processing unit capable of executing instructions, with such instructions stored in volatile or non-volatile memory within or associated with the control circuitry. Control circuitry 110 may also include, operate in conjunction with, or be embedded within an Application Specific Integrated Circuit (ASIC), such as those described earlier. Control circuitry 110 may comprise an integrated circuit with a monocrystalline substrate, or may comprise any number of such integrated circuits operating as a system. Control circuitry may also be included as part of a System-on-Chip (SoC) or a System-on-Module (SoM) which may incorporate memory devices and other digital interfaces. Stimulation circuitry 28 or 58 (FIG. 3) may comprise a portion of the control circuitry 110. Although shown separately, understand that timing channel circuitries 114 can comprise a portion of the control circuitry 110.

The various phases of each waveform period are controlled by timing channels circuitries 114, which operate independently in a given IPG or ETS to enable use of several timing channels. Each timing channel circuitry 114 can concurrently prescribe waveforms that will be formed at electrodes in accordance with the stimulation parameters for the timing channel (e.g., amplitudes, phase durations, frequency, selected anode and cathode electrodes, etc.). The control circuitry 110 typically receives the stimulation parameters for each timing channel wirelessly from an external device, such as the clinician programmer 70 or external controller 60 described earlier (FIG. 5).

FIG. 9C shows further details of a timing channel circuitry 114 used for a single timing channel, such as that useable to form the waveforms 102 described herein. Shown are timer circuitry 112 and a register bank 118. The timer 112 stores the phase durations of the phases in the period, while the register bank 118 stores control information, amplitude, active electrode, and electrode polarity information for the phases. Thus, a first register in the timer 112 stores the phase duration of the first phase in the period (e.g., the pre-pulse phase PDₚₚ), and the corresponding first register in the register bank 118 stores control information (cntlₚₚ) amplitude (Aₚₚ), selected active electrodes, and their polarities (P) for this phase. A second register in the timer 112 stores the phase duration of the next phase (e.g., PDₚ₁), and the corresponding second register in the register bank 118 again stores control information, amplitude, selected active electrodes and polarity for this phase, etc. Data for subsequent phases ip, p2, pr, and q are similarly stored in the timer 112 and register bank 118. The timer 112 may comprise a state machine in one example.

The control data in the registers (cntl) contains information necessary for proper control of the stimulation circuitry 28 or 58 for each phase. For example, during the passive recovery phase pr, the control data (cntlₚᵣ) would instruct one or more passive recovery switches 41ᵢ (FIG. 3) to close, and would disable the various active current-generating PDACs 40ᵢ and NDACs 42ᵢ (FIG. 3). By contrast, during active phases, the control data would instruct the passive recovery switches 41ᵢ to open (be disabled), and would enable relevant PDACs 40ᵢ and NDACs 42ᵢ.

Each register in the register bank 118 is, in one example, 96 bits in length, with the control data for the phase in the first 16 bits, the amplitude of the phase specified in the next 16 bits, followed by eight bits for each electrode (the data for eight electrodes is shown, but this could be varied). The eight electrode bits may include the polarity (P) of the electrode in a single bit (whether the electrode is to act as an anode that sources current or as a cathode that sinks current), with the remaining 7 bits specifying the percentage (%) of the amplitude that that electrode will receive, as explained further in U.S. Pat. No. 9,008,790. Note that during phases where no active current is to be driven by the stimulation circuitry 28 or 58, such as ip, pr, and q, the amplitude of the current may automatically be set to zero. Each register in the timer 112 can store various numbers bits without necessarily deviating from the scope of the present disclosure.

The goal of the timing channel circuitry 114 is to send data from an appropriate register in the register bank 118 to the stimulation circuitry 28 or 58 at an appropriate point in time, and this occurs by control of the timer 112. As noted earlier, the phase durations of the various phases are stored in the timer 112. Also stored at the timer is the frequency, f, the inverse of which (1/f) comprises the duration of each waveform period. Knowing this period, the timer 112 can cycle through each of the phase durations, and send the data in the register bank 118 to the stimulation circuitry 28 or 58 at the appropriate time. Thus, at the start of the period, the timer 112 enables a multiplexer 120 to pass the values stored in the first register for the pre-pulse phase to bus 116, which enables stimulation circuitry 28 or 58 to establish the pre-pulses at the selected electrodes (e.g., E3 and E5 as specified in waveforms 102). After time PDₚₚ has passed, the timer 112 now enables the multiplexer 120 to pass the values stored in the second register for phase p1 to the stimulation circuitry 28 or 58 to establish phase 1 at the selected electrodes. The other registers are similarly controlled by the timer 112 to send the waveform phase data at appropriate times. This process of cycling through the various phases continues, and eventually at the end of quiet phase, i.e., at the end of PD_{q}, the timer 112 once again enables the pre-pulse data, and a new period of waveforms is established.

As noted earlier, the waveforms 102 are preferably formed in a single timing channel, i.e., a single timing channel circuit 114. This is beneficial, because it free ups other timing channel circuits 114 to provide other waveforms if desired. Providing waveforms 102 in a single timing channel also allows arbitration to be enabled in the IPG or ETS. Arbitration, as is known, comprises a means of preventing overlaps in time of the waveforms from different timing channels. Arbitration can operate under the control of arbitration logic (not shown), and when such logic identifies an overlap in time of waveforms from different timing channels it can cause the waveform from one of the timing channels to be delayed to prevent the overlap. See, e.g., U.S. Pat. No. 9,656,081, describing arbitration logic in further detail, which is incorporated herein by reference in its entirety.

While architecture 130 is shown as one manner of making waveforms 102, it should be noted that other current generation architectures can be used as well which have more flexibility in producing waveforms of longer or more-random shapes, such as those disclosed in U.S. Pat. No. 9,008,790 or U.S. Patent Application Publications 2018/0071513 and 2018/0071516, which are incorporated herein by reference in their entireties.

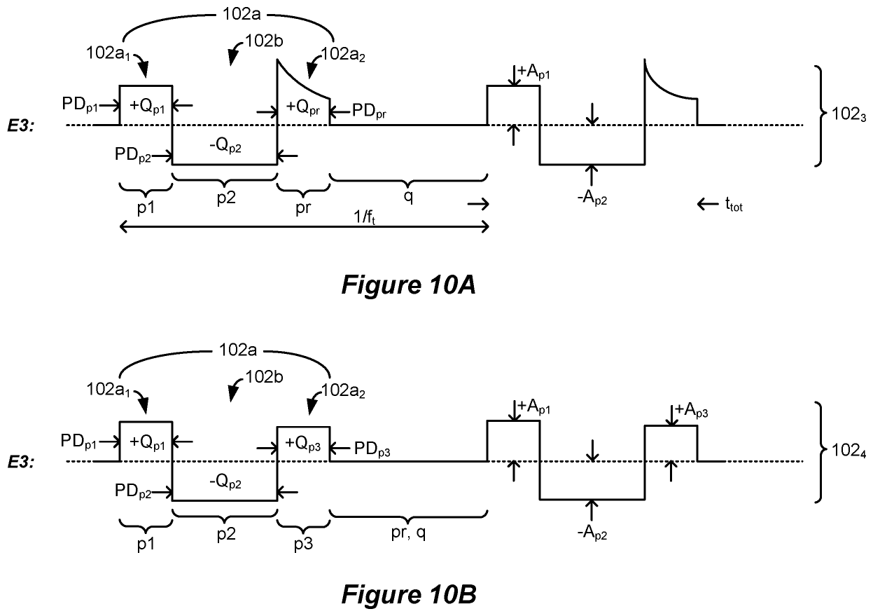

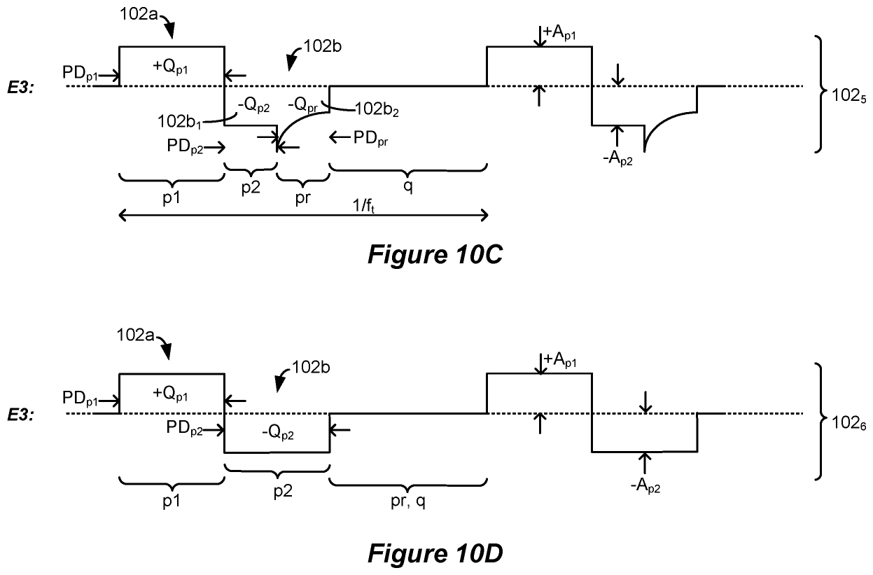

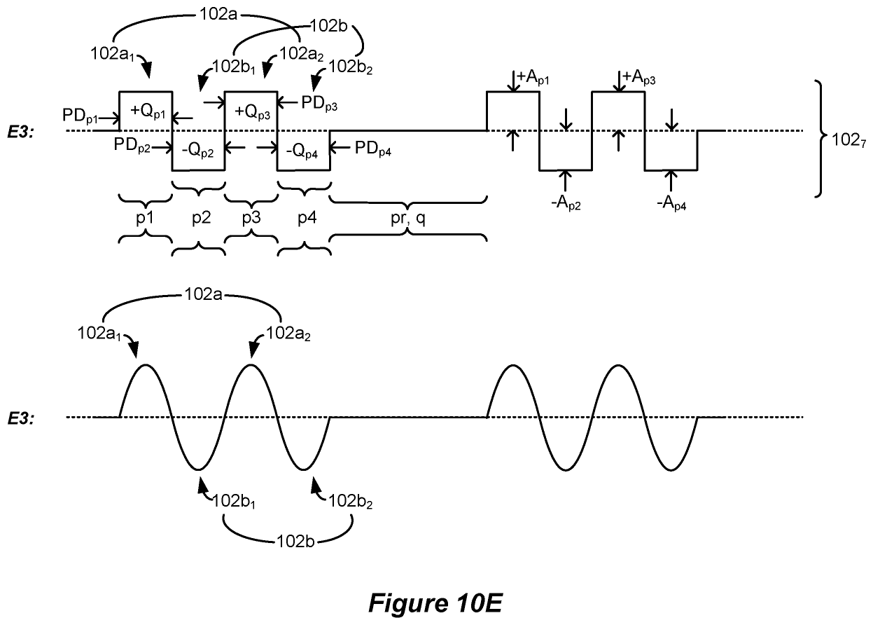

In such other, more-flexible architectures, the waveforms 102 may be formed differently, without restrictions to the number of phases used, or the duration of such phases. FIGS. 10A-10E show examples of other waveforms 102 similar in therapeutic effectiveness to waveforms 102₁ and 102₂ that can be formed using such other architectures. For simplicity, only the waveforms formed at a single electrode (e.g., E3) are shown, although it should be understood that waveforms of opposite polarity (e.g., at E5) would also be involved in providing stimulation to the patient's tissue.

FIGS. 10A and 10B show waveforms 102₃ and 102₄ that are triphasic in nature (like waveforms 102₁) comprising phases 102a and 102b, in which phases 102a are split into sub-phases 102a₁ and 102a₂ of the same polarity, and separated by phase 102b of the opposite polarity.

Starting with waveforms 102₃ in FIG. 10A, sub-phase 102a₁ and phase 102b are actively driven by stimulation circuitry 28 or 58 during arbitrary active phases p1 and p2 that are not specific to any particular current generation architecture circuitry used in the IPG or ETS. Phases p1 and p2 have phase durations PDₚ₁ and PDₚ₂ respectively, and amplitudes +Aₚ₁ and −Aₚ₂ respectively. As with waveforms 102₁, it is assumed that the charge of p1 is lower than the phase p2, i.e., |Qₚ₁|<|Qₚ₂|. Thus, as with waveforms 102₁, passive charge recovery during phase pr, during sub-phase 102a₂, is used to recover charge and to act as a therapeutic phase. At least phase 102b can be formed with a duration of greater than 2.0 ms, or even 2.6 ms or greater, and sub-phases 102a₁ and 102a₂ in total may be similar in duration as well.

For waveforms 102₄ of FIG. 10B, all of sub-phases 102a₁ and 102a₂, and phase 102b, are actively driven by stimulation circuitry 28 or 58 during arbitrary phases p1, p2, and p3, with phase durations PDₚᵣ, PDₚ₂, and PDₚ₃ respectively, and amplitudes +Aₚ₁, −Aₚ₂, and +Aₚ₃ respectively. In this example, the net charge of the various phases p1-p3 at each electrode may equal zero, i.e., |Q₁|+|Qₚ₃|=|Qₚ₂|, such that the waveforms are charge balanced at the end of phase p3. Nonetheless, a passive charge recovery period pr may be used to recover any remaining charge prior to instituting a quiet phase q. Again, at least phase 102b can be formed with a duration of greater than 2.0 ms, or even 2.6 ms or greater, and sub-phases 102a₁ and 102a₂ in total may be similar in duration as well.

FIGS. 10C and 10D show waveforms 102₅ and 102₆ that are biphasic in nature (like waveforms 102₂), and comprising phases 102a and 102b of opposite polarities. In FIG. 10C, showing waveforms 102₅, phase 102a is actively driven by stimulation circuitry 28 or 58 during arbitrary active phase p1 (+Aₚ₁) not specific to any particular current generation architecture. Phase p2 is also actively driven during sub-phase 102b₁ (−Aₚ₂), while its concatenated sub-phase 102b₂ is passively driven during a passive recovery phase pr. Phase 102a has a phase duration PDₚᵣ while phase 102b has a phase duration of PDₚ₂+PDₚᵣ. It is assumed that the charge of p1 is higher than the phase p2, i.e., |Qₚ₁|>|Qₚ₂| to allow sub-phase 102b1 to be formed with the same polarity as sub-phase p2. One or more of phases 102a or 102b can be formed with a duration of greater than 2.0 ms, or even 2.6 ms or greater, which again was shown by earlier simulations to provide a benefit.

In FIG. 10D, showing waveforms 102₆, phases 102a (p1) and 102b (p2) are actively driven during their entire durations and without sub-phases, although each could also include sub-phases to increase the duration of either phase, or to allow the current amplitude of a phase to vary. In the example shown, phases p1 and p2 have phase durations PDₚᵣ and PDₚ₂, and amplitudes +Aₚ₁ and −Aₚ₂. In this example, the phases are charge balanced, i.e., |Qₚ₁|=|Qₚ₂|. Nonetheless, a passive charge recovery period pr may be used to recover any remaining charge prior to instituting a quiet phase q. Again, either or both of phases 102a or 102b can be formed with a duration greater than 2.0 ms, or even 2.6 ms or greater.

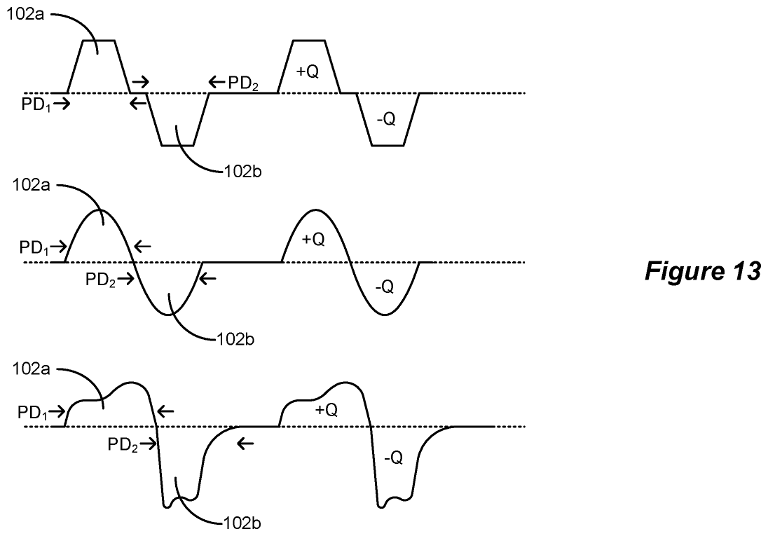

In FIG. 10E, showing waveforms 102₇, phases 102a and 102b are established with actively-driven sub-phases 102a₁ (p2), 102a₂ (p2), 102b₁ (p3), and 102b₂ (p4) which are interleaved, effectively making a quadriphasic waveform. Still further sub-phases (e.g., 102a₃, 102b₃, etc.) could be used as well. Again, either or both of phases 102a or 102b may in total be formed with a duration of greater than 2.0 ms, or even 2.6 ms or greater. FIG. 10E also shows the example of a waveform 102 formed of non-square pulses, in this case a sine wave in which positive currents comprise sub-phases 102a₁ and negative currents comprise sub-phases 102b₁. Waveforms 102 of different shapes are discussed further below with reference to FIG. 13.

Any of the examples of waveforms 102 can be provided to two or more of the electrodes, and in this regard two or more of the electrodes (include the case electrode Ec 12) can act as an anode at one time, and two or more of the electrodes can act as a cathode at one time. An example is shown in FIG. 11, which shows waveforms 102₈ that modify example waveforms 102₁ illustrated earlier, although any example of waveforms 102 could be similarly modified. In this example, only electrode E3 is selected as an anode electrode during sub-phase 102a₁, and thus that electrode will receive 100% of a prescribed current I as an anodic current (+I). However, electrodes E4 and E5 are both selected to operate as cathode electrodes simultaneously, with E4 receiving 70% of I as a cathodic current (−0.7I), and E5 receiving the remaining 30% of I as a cathodic current (−0.3I). Other phases or sub-phases could prescribe different current magnitudes I, with E3, E4, and E5 sharing the same +100%, −70%, and −30% split between each of the phases.

In short, a prescribed current I during any phase or sub-phase can be allocated, or steered, to a plurality of different electrodes as an anodic current (+I) or to a plurality of different electrode as a cathodic current (−I) at any point in time, thus allowing independent current control at the electrodes 16. Current steering between electrodes is further explained in one example in U.S. Patent Application Publication 2019/0083796, which is incorporated herein by reference in its entirety. Choosing more than one electrode to act as an anode or cathode electrode at one time also allows for the creation of virtual poles in the tissue that may not correspond to the physical location of the electrodes 16, and/or may allow for the formation of multipoles (e.g., tripoles) to stimulate a patient's tissue, as explained in U.S. patent application Ser. No. 16/210,814 (the '814 Application), filed Dec. 5, 2018, which is incorporated herein by reference in its entirety. Choosing more than two electrodes to provide stimulation can also be used to shape the resulting electric field in the patient's tissue, or to alter the direction at which such electric field is imparted. This is noteworthy in some examples because adjusting the electrodes or steering the current between them in different manners can focus the electric field towards the dorsal horn, where most IIN cells are theorized to be present, as discussed earlier with reference to FIGS. 6A and 6B. Alternatively, electrode adjustment and current steering can also focus the electric field more toward the excitatory neurons in the dorsal column, as discussed earlier with reference to FIG. 7. Note further that when selecting one or more of the anode electrodes, or one or more of the cathode electrodes, one of these selected electrodes can comprise the case electrode Ec 12 (FIG. 3).

As noted earlier, the various examples of waveforms 102 shown above may be charge balanced at each electrode, but this is not strictly required as shown in FIG. 12. In some cases, perfect charge recovery may not be ideal, and this may especially be true for waveforms with longer phase durations. Every time a waveform is issued, electrochemical reactions occur at the electrode/tissue interface. Most of these reactions are reversible and the electric charge can be recovered. However, some of these reactions may not be reversible, meaning that the charge involved in those reactions is not recoverable. Forcing the system to recover 100% of the charge could therefore inadvertently cause additional reactions in an effort to recover non- recoverable charge, and the amount of non-recoverable charge would increase with longer phase durations. Therefore, the use of charge imbalanced waveforms 102 can be beneficial in some circumstances.

Charge imbalanced waveforms 102 can also be beneficial to cause a pseudo- constant DC current to flow in the tissue. FIG. 12 shows an example of waveforms 102₉ again comprising phases 102a and 102b, either of which may comprise sub-phases, although this isn't shown. The waveforms at E3 and E5 are charge imbalanced in that the phases 102a and 102b don't comprise the same amount of charge. Specifically, the waveforms at E3 have positive phases 102a with a charge (+Qs) that is smaller than the larger negative charge of phases 102b (i.e., |+Qs|<|−Q1|). Each waveform at E3 therefore has a net negative charge. This is reversed at return electrode E5, which has a net positive charge. As explained in the '814 Application, this causes charge to build on capacitances in the current path between the electrodes, and in particular across capacitors C3 and C5, resulting in negative voltages Vc3 and Vc5. This causes a voltage Vdc to build from electrode E3 to E5 during the quiet periods between the waveforms. Vdc in turn causes a pseudo constant DC current, Idc, to flow from E3 to E5, where Idc=Vdc/R, and where R equals the resistance of the tissue between the electrodes. As explained in the '814 Application, using charge imbalanced waveforms to cause a background current Idc to flow can be useful during neurostimulation, because it can affect neuron polarization, changes in synaptic efficacy, or even DC conduction block. The '814 Application discusses other variations not repeated here, as well as aspects that can be affected at the GUI 82 of the clinician programmer 70 (FIG. 5) to assist in the formation of charge imbalanced waveforms.

There are many ways in which the various examples of waveforms 102 shown above could be modified in an actual implementation while still providing good therapeutic effects. As mentioned earlier, the amplitudes provided during phases 102a and 102b, or their sub-phases (e.g., 102a₁ and 102a₂) could be of differing amplitudes, and it is not necessary that any phase or sub-phase have a current amplitude that is constant over its phase duration. In other words, the phases or sub-phases can have random shapes (e.g., sine waves, trapezoids, triangles, sawtooth waves, etc.). FIG. 13 shows examples of trapezoidal and sine wave waveforms, as well as a waveforms of a random shape. In this example, the waveforms are all charge balanced with phase 102a (+Q) being equal but of opposite polarity to phase 102b (−Q), because the time integral of the current (Q=INT I(t) dt), or said more simply the area under the curves, is the same for each of the phases. Note that the GUI 82 of the clinician programmer 70 (FIG. 5) can provide options to vary the waveforms 102 in these and other manners.

Actively-driven phases need not involve driving the electrodes with a constant current as occurs when current sources PDAC(s) 40ᵢ and NDAC(s) 42ᵢ are used in the stimulation circuitry 28 or 58. Phases may also be actively driven by voltage sources, or using sources having capacitors that are pre-charged and then discharged to provide a non-constant (e.g., exponentially decaying) stimulation current to the tissue.

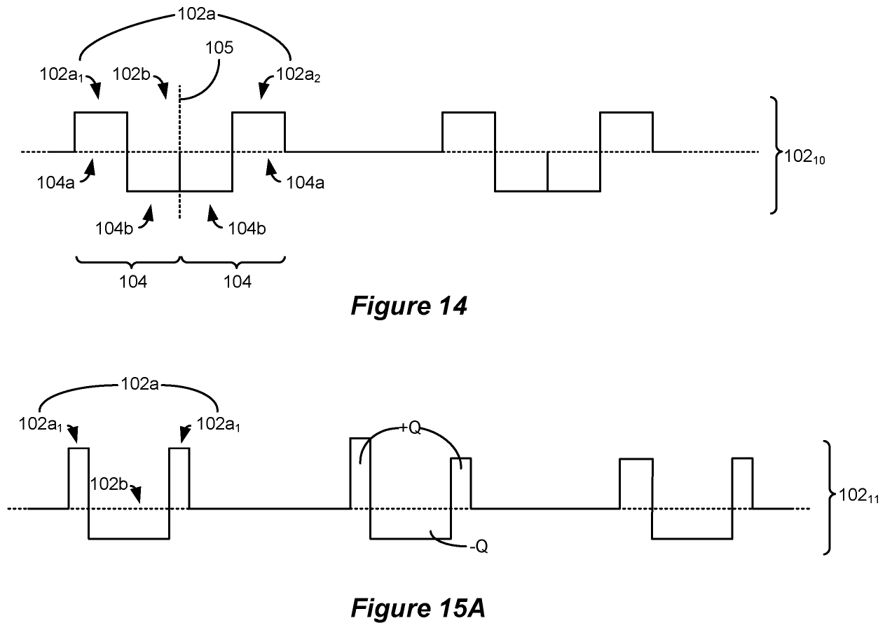

FIG. 14 shows another manner in which waveforms 102 may be formed. In this example, long-duration waveforms 102₁₀ are formed by mirroring two waveforms 104 across an axis 105. Waveforms 104 are biphasic having phases 104a and 104b, and thus when mirrored create a triphasic waveform 102₁₀ having phases 102a and 102b, in which phase 102a is broken into two sub-phases 102a₁ and 102a₂ as occurred in earlier examples. As before, either or both of phase 102a (in total) or 102b can be formed with a duration of greater than 2.0 ms, or even 2.6 ms or greater. Note that a negligible delay may occur between the issuance of the mirrored waveforms 104, amounting to a small gap (100 microsecond or less) at the axis 105.

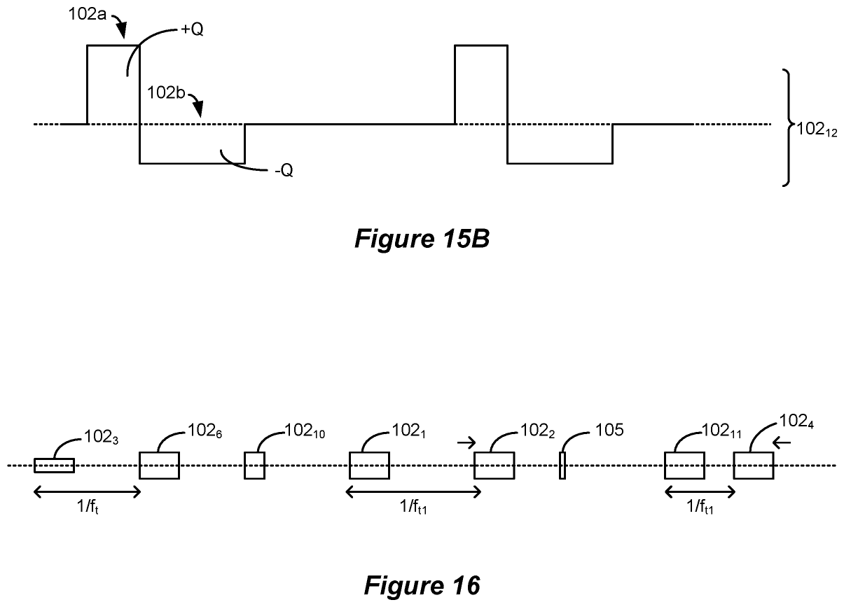

FIGS. 15A and 15B show how charge balanced waveforms 102₁₁ can be formed having different amplitudes or phase durations between the phases. FIG. 15A shows triphasic waveforms as already described. However, in the first waveform 102₁₁, the amplitude of the currents during sub-phases 102a₁ and 102a₂ are different (higher) from the amplitude used during phase 102b. To keep charge balance, note that the phase duration of sub-phases 102a₁ and 102a₂ have been made shorter. In the second waveform, the amplitudes of the sub-phases 102a₁ and 102a₂ are different, and both are different from the amplitude used during phase 102b. In the third waveform, the amplitudes of the sub-phases 102a₁ and 102a₂ are the same, but their phase durations differ. Still, in all of these examples, the resulting waveform 102₁₁ has at least one phase (102b) which is formed with a duration of 2.0 ms or greater, or even 2.6 ms or greater. FIG. 15B shows biphasic charge-balanced waveforms 102₁₂ which unlike earlier examples are not symmetric between phases 102a and 102b. Specifically, phase 102a has a higher amplitude but a shorter phase duration when compared with phase 102b. Again though, the resulting waveform 102₁₂ has at least one phase (102a or 102b) which can be formed with a duration of greater than 2.0 ms, or even 2.6 ms or greater.

The various waveforms 102 can be varied and need not comprise a periodic issuance of the same waveform, and need not comprise a constant tonic frequency fₜ. This is shown in FIG. 16, which show essentially a random issuance of the example waveforms (102₁, 102₂, etc.) described earlier, and which illustrates that the waveforms 102 may generally be mixed and matched in any way or pattern. Further, the waveforms 102 need not issue at a constant tonic frequency fₜ, as shown by the later issuance of waveform 102₂ (fₜ₁) and the earlier issuance of waveform 102₄ (fₜ₂) in FIG. 16. Other variations may be used as well. For example, the amplitudes, phase durations, or tonic frequency fₜ of the waveforms may increase or decrease to ease therapy in or out. For example, first waveform 102₃ is generally of a smaller amplitude, and indicates how waveforms 102 can be ramped up at the start of simulation to ease in therapy. Waveform 102₁₀ has smaller phase durations when compared to the other waveforms, etc. Waveforms 102 with long pulse widths may also be mixed with other waveforms, such as 105, which may have shorter durations. Such variations in the waveforms 102 can be affected by the GUI 84 of the clinician programmer 70.

The long phase durations may be automatically configured in the clinician programmer 70 (FIG. 5) by reference to properties of known neural targets, such as the IIN cells or dorsal column axons. For example, the clinician programmer 70 may store information of strength-duration curves (SDC) for such neural targets. This information can allow the clinician programmer 70 to automatically set the phase durations as needed for a given amplitude, so that a chronaxie time multiple is established, or to ensure that the stimulation will be greater that the rheobase of the neural target. The GUI 84 of the clinician programmer 70 may also display to the user if a phase duration exceeds SDC-based duration estimates, or phase durations may be pre-defined according to these predictions.

To summarize, methods of providing stimulation and stimulators in accordance with the disclosed technique may provide waveforms with first and second phases such as 102a and 102b in which at least one of the phases has a duration of greater than 2.0 ms, noticed to be effective via simulation (see FIG. 6B), or in which at least one of the phases has a duration of 2.6 ms or greater which is not supported by traditional SCS systems. An upper limit of such long phase durations can vary depending on the application at hand, and with reference to the neural target and the amplitudes used for stimulation. In different examples, the phase durations may have an upper limit of 10 ms, 20 ms, 50 ms, 100 ms, 200 ms, or 500 ms.

Long phase durations as disclosed herein may also be applied to other neuronal tissues, such as peripheral nerves, auditory nerves, cochlear cells, retina cells, olfactory cells, nerve roots, ganglia, brain tissue as useful in Deep Brain Stimulation (DBS), autonomic fibers including those innervating organs and smooth muscles, efferent and afferent nerves, muscle tissue, descending axons from supraspinal pain modulatory centers and their branch points and terminals, A-fiber and non-nociceptive C-fiber afferent terminals, and non-neuronal spinal elements such as astrocytes and microglia.

Although particular embodiments of the present invention have been shown and described, it should be understood that the above discussion is not intended to limit the present invention to these embodiments. It will be obvious to those skilled in the art that various changes and modifications may be made without departing from the spirit and scope of the present invention. Thus, the present invention is intended to cover alternatives, modifications, and equivalents that may fall within the spirit and scope of the present invention as defined by the claims.

What is claimed is:

1. A method for providing stimulation to a patient, comprising:
providing a plurality of electrodes of a spinal cord stimulator proximate to a patient's spinal cord;
selecting at least one electrode to recruit neural elements of the patient's spinal cord; and
providing, from stimulation circuitry in the spinal cord stimulator, waveforms to the selected at least one electrode to cause stimulation of the patient's spinal cord, wherein the waveforms comprise a first phase of a first polarity during a first duration and a second phase of a second polarity opposite the first polarity during a second duration following the first duration,
wherein the first duration is greater than 2.0 ms and less than 500 ms, and
wherein the first phase lacks a quiescent period during which no stimulation is provided from the stimulation circuitry to the patient's spinal cord.

2. The method of claim 1, wherein the first duration is 2.6 ms or greater.

3. The method of claim 1, wherein the first duration is 10 ms or less.

4. The method of claim 1, wherein the second duration is greater than 2.0 ms and less than 500 ms, and wherein the second phase lacks a quiescent period during which no stimulation is provided from the stimulation circuitry to the patient's spinal cord.

5. The method of claim 4, wherein the second duration is 2.6 ms or greater.

6. The method of claim 4, wherein the second duration is 10 ms or less.

7. The method of claim 1, wherein at least one of the first phase or the second phase comprises concatenated first and second sub-phases, wherein one of the sub-phases is actively driven with a current by the stimulation circuitry, and wherein the other of the sub-phases is passively driven by the stimulation circuitry.

8. The method of claim 1, wherein the first and second phases are actively driven with a current by the stimulation circuitry over their respective entireties.

9. The method of claim 1, wherein the first and second phases are charge balanced at each of the at least one electrodes.

10. The method of claim 1, wherein the first and second phases are not charge balanced at each of the at least one electrodes for at least some of the waveforms.

11. The method of claim 1, wherein the waveforms are provided to the selected at least one electrode at one or more frequencies comprising 60 Hz or less.

12. The method of claim 1, wherein the at least one electrode is selected to selectively recruit neural elements in a dorsal horn of the patient's spinal cord.

13. The method of claim 12, wherein the stimulation comprises sub-perception stimulation.

14. The method of claim 13, wherein at least one of the first phase or the second phase comprises a current amplitude of 0.6 mA or less over its respective entire duration.

15. The method of claim 1, wherein the at least one electrode is selected to selectively recruit neural elements in a dorsal column of the patient's spinal cord.

16. The method of claim 15, wherein the stimulation comprises supra-perception stimulation.

17. The method of claim 16, wherein at least one of the first phase or the second phase comprises a current amplitude of greater than 0.6 mA over its respective entire duration.

18. The method of claim 15, wherein the stimulation comprises sub-perception stimulation but above a dorsal column activation threshold.

19. The method of claim 1, wherein stimulation parameters for the waveforms are provided to the stimulation circuitry by a single timing channel circuitry of the spinal cord stimulator.

20. The method of claim 1, wherein the selected at least one electrode comprises one or more anodic electrodes and one or more cathodic electrodes.
